(19) United States
(12) Patent Application Publication
(10) Pub. No.: US 2025/0345597 A1
(43) Pub. Date: Nov. 13, 2025

(54) APICAL INNER EAR STIMULATION

(71) Applicant: Cochlear Limited, Macquarie University (AU)

(72) Inventors: Christopher Joseph Long, Centennial, CO (US); Zachary Mark Smith, Pymble (AU)

(21) Appl. No.: 19/276,128

(22) Filed: Jul. 22, 2025

Related U.S. Application Data
(63) Continuation of application No. 17/294,120, filed on May 14, 2021.
(60) Provisional application No. 62/833,825, filed on Apr. 15, 2019.

(51) Int. Cl. A61N 1/05; A61N 1/36

(52) U.S. Cl. CPC A61N 1/0541; A61N 1/36038; A61N 1/36128

(57) ABSTRACT

An apical cochlear implant comprises an apical electrode assembly in combination with a basilar electrode assembly. The apical cochlear implant is configured to stimulate a cochlear of a recipient via the apical electrode assembly in combination with the basilar electrode assembly.

### BACKGROUND

### Field of the Invention

The present invention generally relates to apical inner ear stimulation.

### Related Art

Hearing loss, which may be due to many different causes, is generally of two types, conductive and/or sensorineural. Conductive hearing loss occurs when the normal mechanical pathways of the outer and/or middle ear are impeded, for example, by damage to the ossicular chain or ear canal. Sensorineural hearing loss occurs when there is damage to the inner ear, or to the nerve pathways from the inner ear to the brain.

Individuals who suffer from conductive hearing loss typically have some form of residual hearing because the hair cells in the cochlea are undamaged. As such, individuals suffering from conductive hearing loss typically receive an auditory prosthesis that generates motion of the cochlea fluid. Such auditory prostheses include, for example, acoustic hearing aids, bone conduction devices, and direct acoustic stimulators.

In many people who are profoundly deaf, however, the reason for their deafness is sensorineural hearing loss. Those suffering from some forms of sensorineural hearing loss are unable to derive suitable benefit from auditory prostheses that generate mechanical motion of the cochlea fluid. Such individuals can benefit from implantable auditory prostheses that stimulate nerve fibers of the recipient's auditory system in other ways (e.g., electrical, optical and the like). Cochlear implants are often proposed when the sensorineural hearing loss is due to the absence or destruction of the cochlea hair cells, which transduce acoustic signals into nerve impulses. An auditory brainstem stimulator is another type of stimulating auditory prosthesis that might also be proposed when a recipient experiences sensorineural hearing loss due to damage to the auditory nerve.

### SUMMARY

In one aspect, an apical cochlear implant is provided. The apical cochlear implant comprises: a basilar electrode assembly comprising a plurality of electrodes, wherein the basilar electrode assembly is configured to be implanted into a cochlea of a recipient via a basal region of the cochlea; an apical electrode assembly comprising a plurality of apical electrodes, wherein the apical electrode assembly is dimensioned so as to be implanted within an apical region of the cochlea; one or more sound input devices configured to receive sound signals; a sound processing module configured to convert the sound signals into stimulation control signals; and a stimulator unit configured to generate and deliver, based on the stimulation control signals, a plurality of stimulation signals to the cochlea of the recipient via the basilar electrode assembly and the apical electrode assembly.

In another aspect, a method is provided. The method comprises: receiving sound signals at one or more sound input devices of a cochlear implant, wherein the cochlear implant comprises an apical electrode assembly comprising a plurality of apical electrodes and a basilar electrode assembly comprising a second plurality of electrodes; generating a plurality of stimulation signals representative of the sound signals; directly delivering, via one or more of the plurality of apical electrodes, a first subset of the plurality stimulation signals to a first tonotopic region of the cochlea, wherein the first tonotopic region is associated with acoustic frequencies below a predetermined threshold frequency; and directly delivering, via one or more of the second plurality of electrodes of the basilar electrode assembly, a second subset of the plurality of stimulation signals to a second tonotopic region of the cochlea.

In another aspect, an apparatus is provided. The apparatus comprises: a basilar electrode assembly comprising a plurality of electrodes; an apical electrode assembly comprising a plurality of apical electrodes; one or more sound input devices configured to receive sound signals; a sound processing module configured to convert the sound signals into stimulation control signals; and a stimulator unit configured to: generate, based on the stimulation control signals, a plurality of stimulation signals; directly stimulate, via one or more of the plurality of electrodes of the basilar electrode assembly, a high frequency region of the cochlea; and directly stimulate, via one or more of the plurality of apical electrodes, a low frequency region of the cochlea.

### BRIEF DESCRIPTION OF THE DRAWINGS

Embodiments of the present invention are described herein in conjunction with the accompanying drawings, in which:

FIG. 1A is a partially cut-away, perspective view of a cochlea in which an apical cochlea electrode assembly can be implanted;

FIG. 1B is a cross-sectional view of one turn of the canals of the cochlea of FIG. 1A;

FIG. 2A is a schematic diagram illustrating an apical cochlear implant, in accordance with certain embodiments presented herein;

FIG. 2B is a block diagram of the apical cochlear implant of FIG. 2A;

FIG. 2C is a schematic diagram illustrating further details of an apical cochlea electrode assembly and a basilar electrode assembly of FIG. 2A implanted in a cochlea of a recipient, in accordance with certain embodiments presented herein;

FIG. 3 is a graph illustrating changes in cross-sectional area of example cochleas;

FIG. 4 is a functional block diagram illustrating operation of a sound processing module of an apical cochlear implant, accordance with certain embodiments presented herein;

FIGS. 5A and 5B are schematic diagrams illustrating focused channel configurations, in accordance with certain embodiments presented herein;

FIG. 6A is a graph illustrating threshold amplitudes relative to angular cochlea depth for monopolar stimulation delivered via a basilar electrode assembly, where the return current is sunk via an extra-cochlear electrode;

FIG. 6B is a graph illustrating threshold amplitudes relative to angular cochlea depth for biphasic stimulation delivered via a basilar electrode assembly, where the return current is sunk via one or more electrodes implanted in an apical region of the cochlea;

FIG. 6C is a graph illustrating threshold amplitudes relative to angular cochlea depth for partial bipolar stimulation delivered via a basilar electrode assembly, where the return current is sunk via one or more electrodes implanted in an apical region of the cochlea and an extra-cochlear electrode, in accordance with certain embodiments presented herein;

FIG. 7 is a functional block of a sound processing module of an apical cochlear implant to optimize speech/sound understanding through use of an apical electrode assembly, in accordance with certain embodiments presented herein;

FIG. 8 is a functional block of a sound processing module of an apical cochlear implant to optimize speech/sound understanding through use of an apical electrode assembly, in accordance with certain embodiments presented herein;

FIG. 9 is a functional block of a sound processing module of an apical cochlear implant to optimize speech/sound understanding through use of an apical electrode assembly, in accordance with certain embodiments presented herein;

FIG. 10 is a functional block of a sound processing module of an apical cochlear implant to optimize speech/sound understanding through use of an apical electrode assembly, in accordance with certain embodiments presented herein;

FIG. 11 is a schematic diagram illustrating pulse trains, in accordance with certain embodiments presented herein;

FIG. 12 is a schematic diagram illustrating pulse trains, in accordance with certain embodiments presented herein; and

FIG. 13 is a high-level flowchart of a method, in accordance with certain embodiments presented herein.

### DETAILED DESCRIPTION

A recipient's cochlea is sometimes referred to as having an “apical” or “distal” region and a “basal” or “proximal” region. For ease of description, an electrode assembly implanted in, configured to be implanted in, or configured to be implanted via the apical region of a recipient's cochlea is referred to herein as an “apical cochlea electrode assembly” or, more simply, an “apical electrode assembly.” Additionally, also for ease of description, an electrode assembly implanted in, configured to be implanted in, or configured to be implanted via the basal region of a recipient's cochlea is referred to herein as a “basal cochlea electrode assembly” or, more simply, a “basilar electrode assembly.” Presented herein are techniques for stimulating a cochlear of a recipient with an apical electrode assembly in combination with a basilar electrode assembly.

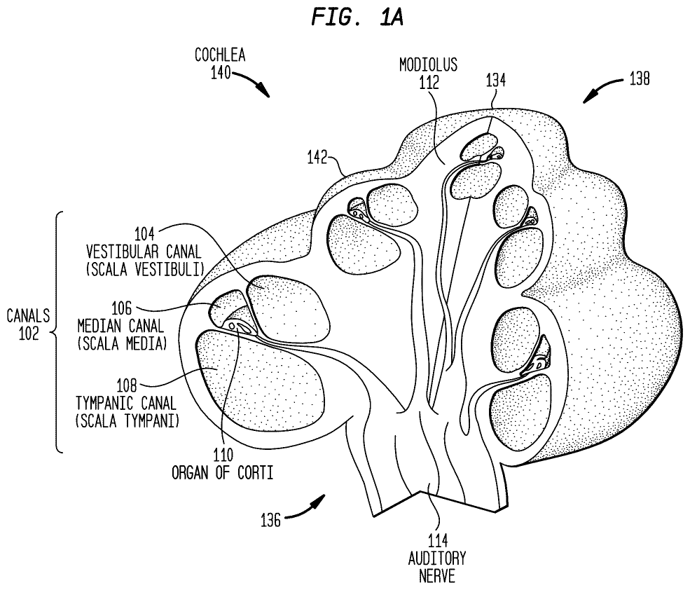

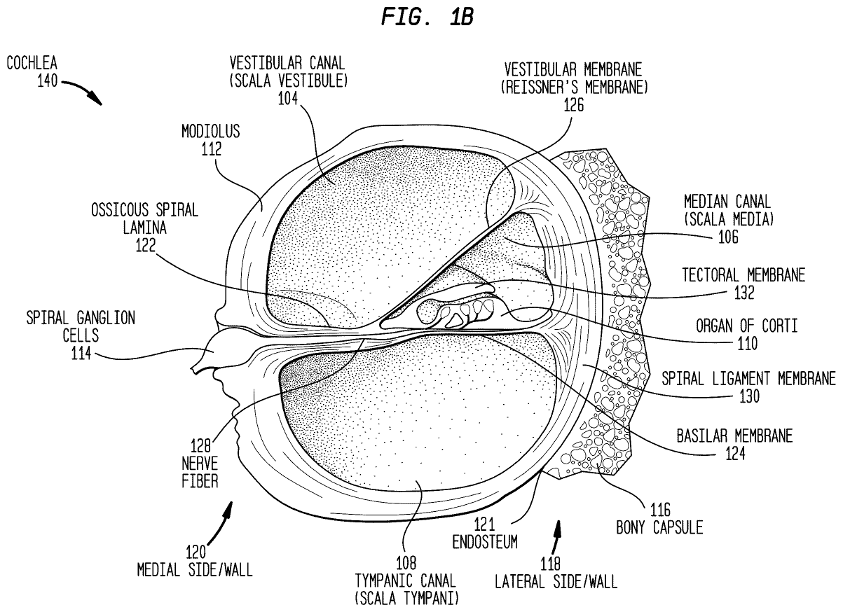

Before describing details of the techniques presented herein, relevant aspects of an example cochlea 140 in which an apical electrode assembly may be implanted are first described below with reference to FIGS. 1A-1B. More specifically, FIG. 1A is a perspective view of the cochlea 140 partially cut-away to display the canals and nerve fibers of the cochlea, while FIG. 1B is a cross-sectional view of one turn of the canals of the cochlea 140.

Referring first to FIG. 1A, cochlea 140 is a conical spiral structure comprising three parallel fluid-filled canals or ducts, collectively and generally referred to herein as canals 102. Canals 102 comprise the tympanic canal 108, also referred to as the scala tympani 108, the vestibular canal 104, also referred to as the scala vestibuli 104, and the median canal 106, also referred to as the scala media 106. Cochlea 140 spirals about modiolus 112 several times and terminates at cochlea apex 134.

Portions of cochlea 140 are encased in a bony labyrinth/capsule 116 and the endosteum 121 (e.g., a thin vascular membrane of connective tissue that lines the inner surface of the bony tissue that forms the medullary cavity of the bony labyrinth). Spiral ganglion cells 114 reside on the opposing medial side 120 (the left side as illustrated in FIG. 1B) of cochlea 140. A spiral ligament membrane 130 is located between lateral side 118 of spiral tympani 108 and bony capsule 116, and between lateral side 118 of scala media 106 and bony capsule 116. Spiral ligament 130 also typically extends around at least a portion of lateral side 118 of scala vestibuli 104.

The fluid in the tympanic canal 108 and the vestibular canal 104, referred to as perilymph, has different properties than that of the fluid which fills scala media 106 and which surrounds organ of Corti 110, referred to as endolymph. The tympanic canal 108 and the vestibular canal 104 collectively form the perilymphatic fluid space 109 of the cochlea 140. Sound entering a recipient's auricle (not shown) causes pressure changes in cochlea 140 to travel through the fluid-filled tympanic and vestibular canals 108, 104. As noted, the organ of Corti 110 is situated on basilar membrane 124 in the scala media 106 and contains rows of 16,000-20,000 hair cells (not shown) which protrude from its surface. Above them is the tectoral membrane 132 which moves in response to pressure variations in the fluid-filled tympanic and vestibular canals 108, 104. Small relative movements of the layers of membrane 132 are sufficient to cause the hair cells in the endolymph to move thereby causing the creation of a voltage pulse or action potential which travels along the associated nerve fiber 128. Nerve fibers 128, embedded within the spiral lamina 122, connect the hair cells with the spiral ganglion cells 114 which form auditory nerve 114. Each cell in these nerve fibers 128 emit a peripheral process that extends toward the organ of Corti 110 and a central process that projects into the auditory nerve 114. Auditory nerve 114 relays the impulses to the auditory areas of the brain (not shown) for processing.

The place along basilar membrane 124 where maximum excitation of the hair cells occurs determines the perception of pitch and loudness according to the place theory. Due to this anatomical arrangement, cochlea 140 has characteristically been referred to as being “tonotopically mapped.” That is, regions of cochlea 140 toward basal region 136 are responsive to high frequency signals, while regions of cochlea 140 toward apical region 138 are responsive to low frequency signals (i.e., low frequency tonotopic regions and high frequency tonotopic regions). These tonotopical properties of cochlea 140 are exploited in a cochlear implant by delivering stimulation signals within a predetermined frequency range to a region of the cochlea that is most sensitive to that particular frequency range.

In general, the basal region 136 is the portion of the cochlea 140 located closest to the stapes (not shown in FIGS. 1A and 1B) and extends to approximately the first turn of the cochlea (i.e., the region of the cochlea 140 between the cochlea openings, including the round and oval windows, the first cochlea turn). The apical region 138 is the portion of the cochlea 140 in proximity to the cochlear apex 134. More specifically, the cochlea 140 is generally a conical spiral structure (i.e., the spiral-like shape) and the apical region 138 of the cochlea 140 is generally the last/final (i.e., most apical) 360 degrees of the cochlea and encompasses the cochlea areas tonotopically associated with hair cells and peripheral processes tuned to frequencies below 0.5 kilohertz (kHz).

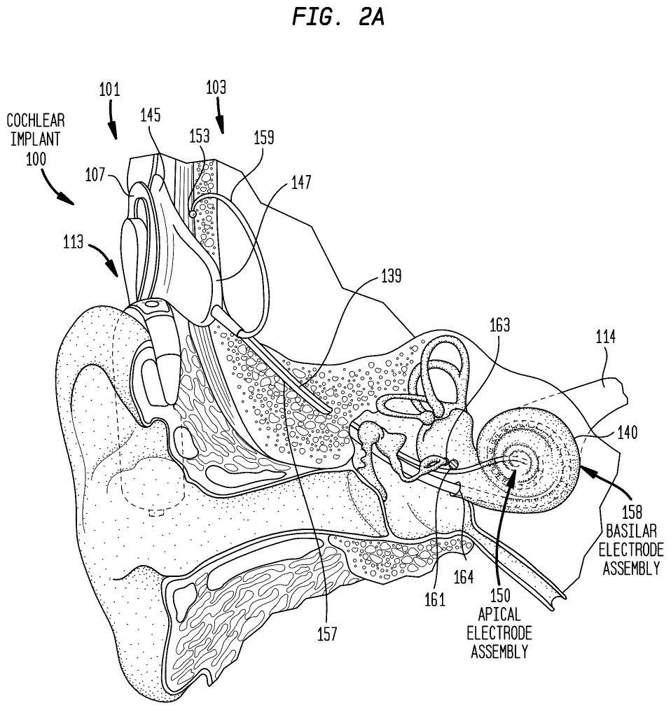

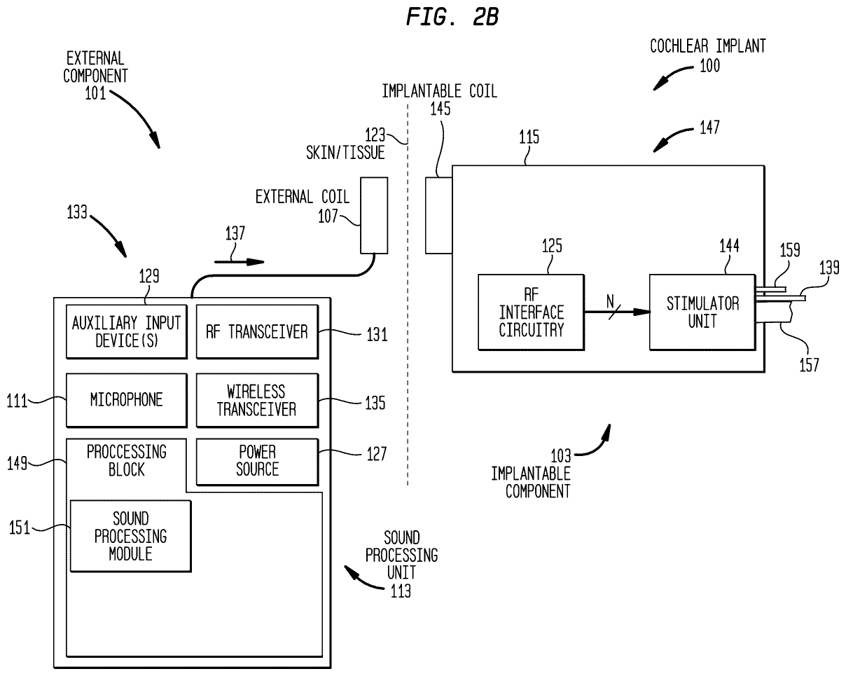

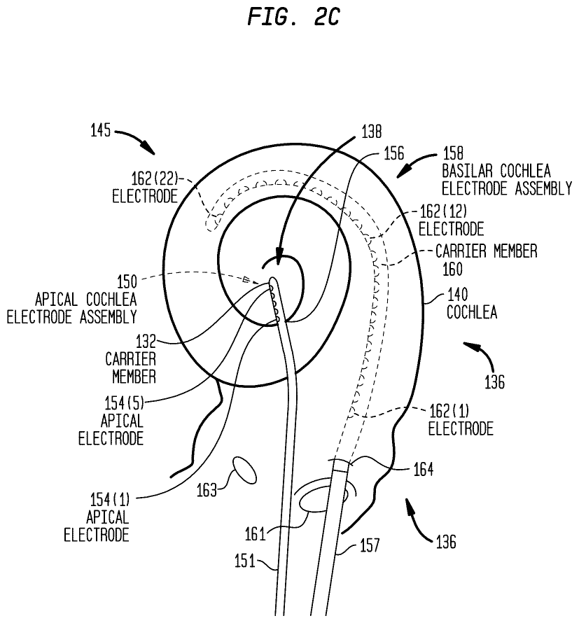

FIG. 2A is a schematic diagram of an exemplary cochlear implant 100 configured to implement aspects of the techniques presented herein, while FIG. 2B is a block diagram of the cochlear implant 100. FIG. 2C is schematic diagram illustrating further details of a portion of the cochlear implant 100. For ease of description, FIGS. 2A, 2B, and 2C will be described together and with reference to implantation of a portion of the cochlear implant 100 into cochlea 140 of FIGS. 1A and 1B.

The cochlear implant 100 comprises an external component 101 and an internal/implantable component 103. The external component 101 is directly or indirectly attached to the body of the recipient and typically comprises an external coil 107 and, generally, a magnet (not shown in FIG. 2A) fixed relative to the external coil 107. The external component 101 also comprises one or more input elements/devices 133 for receiving input signals at a sound processing unit 113. In this example, the one or more input devices 133 include a plurality of microphones 111 (e.g., microphones positioned by the auricle of the recipient, telecoils, etc.) configured to capture/receive input acoustic/sound signals (sounds), one or more auxiliary input devices 129 (e.g., a telecoil, one or more audio ports, such as a Direct Audio Input (DAI), a data port, such as a Universal Serial Bus (USB) port, cable port, etc.), and a wireless transmitter/receiver (transceiver) 135, each located in, on, or near the sound processing unit 113.

The sound processing unit 113 also includes, for example, at least one battery 127, a radio-frequency (RF) transceiver 131, and a processing block 149. The processing block 149 comprises a number of elements, including a sound processing module 151. The sound processing module 151 and may be formed by one or more processors (e.g., one or more Digital Signal Processors (DSPs), one or more uC cores, etc.), firmware, software, etc. arranged to perform operations described herein. That is, the sound processing module 151 may be implemented as firmware elements, partially or fully implemented with digital logic gates in one or more application-specific integrated circuits (ASICs), partially or fully in software, etc.

The implantable component 103 comprises an implant body (main module) 147, an apical electrode assembly 150, and a basilar electrode assembly 158 each configured to be implanted under the skin/tissue (tissue) 123 of the recipient. Since cochlear implant 100 includes both an apical cochlea electrode assembly 150 and a basilar electrode assembly 158, the cochlear implant is sometimes referred to herein as an “apical cochlear implant” 100.

The implant body 147 generally comprises a hermetically-sealed housing 115 in which RF interface circuitry 125 and a stimulator unit 144 are disposed. The implant body 147 also includes an internal/implantable coil 145 that is generally external to the housing 115, but which is connected to the RF interface circuitry 125 via a hermetic feedthrough (not shown in FIG. 2B).

The apical electrode assembly 150 comprises a plurality of apical electrodes 154 disposed in a carrier member 152 (e.g., a flexible silicone body). In this specific example, the apical electrode assembly 150 comprises five (5) apical electrodes, referred to as apical electrodes 154(1), 154(2), 154(3), 154(4), and 154(5). Each of the apical electrodes 154(1)-154(5) is electrically connected to the stimulator unit 144 via one or more wires (not shown in FIGS. 2A-2C) extending through a lead 139. As a result, the apical electrodes 154(1)-154(5) represent at least five different stimulation channels. It is to be appreciated that this specific embodiment with five apical electrodes is merely illustrative and that the techniques presented herein may be used with one other numbers of apical electrodes implanted into a recipient's cochlea.

As described further below, the positioning of the apical electrodes 154(1)-154(5) within the apical region 138 of the cochlea 140 enables direct stimulation of the low frequency (apical) peripheral processes of the nerve fibers 128 located in the apical region of the cochlea. In accordance with embodiments presented herein, the cochlea 140 is generally described herein as being comprised of four (4) different areas/regions that are each associated with (i.e., responsive to) a different tonotopic frequency range/band. In particular, cochlea 140 is first described herein as having a “low frequency” region, which includes the peripheral processes associated with low frequency nerve fibers. As used herein, the low frequency region of the cochlea includes peripheral processes corresponding to frequencies below a predetermined threshold frequency of approximately 1 kilohertz (kHz) (i.e., the low frequency region of the cochlea is the region generally responsive to frequencies below about 1 kHz).

Cochlea 140 is also described herein as having an “ultra-low” or “very-low” frequency region, which includes the peripheral processes associated with ultra-low frequency nerve fibers. As used herein, the ultra-low frequency region of the cochlea includes peripheral processes corresponding to frequencies below a predetermined threshold frequency of approximately 0.5 kHz (i.e., the ultra-low frequency region of the cochlea is the region generally responsive to frequencies below about 500 Hz).

Cochlea 140 is further described herein as having a “mid frequency” region, which includes the peripheral processes associated with mid frequency nerve fibers. As used herein, the mid frequency region of the cochlea includes peripheral processes corresponding to frequencies above approximately 1 kHz, but below approximately 2 kHz (i.e., the mid frequency region of the cochlea is the region generally responsive to frequencies between about 1 kHz and about 2 kHz).

Finally, cochlea 140 is described herein as having a “high frequency” region, which includes the peripheral processes associated with high frequency nerve fibers. As used herein, the high frequency region of the cochlea includes peripheral processes corresponding to frequencies above approximately 2 kHz (i.e., the high frequency region of the cochlea is the region generally responsive to frequencies above about 2 kHz).

In accordance with embodiments presented herein, the four different regions of the cochlea 140 (i.e., the ultra-low frequency, low frequency, mid frequency, and high frequency regions) are not arbitrary boundaries, but instead correlate with different physical characteristics, namely different temporal precision (temporal coding capabilities). More specifically, it has been determined that the precision of acoustic phase locking decreases with frequencies above approximately 1 to 2 kHz and normal hearing acoustic pitch discrimination abilities deteriorate above 2kHz. Moreover, for acoustic stimulation, higher “vector strengths” are found at and below characteristics frequencies (CFs) of 1 kHz and 2 kHz, where higher vector strength indicate better temporal coding. For example, at a characteristic frequency of 1 kHz, the vector strength may be 0.5, while at a characteristic frequency of 2 kHz, the vector strength may be 0.1. A linear decrease in vector strength between 1 kHz and 2 kHz has been reported, with minimal temporal coding above approximately 2 kHz.

In addition, the auditory nerve follows higher rates of electrical stimulation at lower characteristics frequencies. For example, electrical stimulation rates greater than approximately 450 pps are well followed by auditory nerves with characteristic frequencies below approximately 1 kHz, an electrical stimulation rate of approximately 300 pps is well followed at a characteristic frequency of approximately 2 kHz, whereas an electrical stimulation rate of approximately 200 pps is only well followed at a characteristic frequency of greater than approximately 4 kHz. Furthermore, fundamental frequency of the human voice occurs mostly in the tonotopic regions below 500 Hz.

Therefore, as is clear from the above, the boundaries defining the ultra-low frequency, low frequency, mid frequency, and high frequency regions of cochlea 140 are non-arbitrary. Instead, the boundaries defining the different regions correlate with changes in physical characteristics of the auditory nerves of the cochlea 140, namely different temporal coding capabilities.

In accordance with embodiments presented herein, the stimulation delivered by the apical electrodes 154(1)-154(5) is sometimes referred to herein as “direct” low frequency stimulation (direct low frequency stimulation channels or low frequency electrodes) because the apical electrodes 154(1)-154(5) are positioned in close proximity to the target low frequency (apical) peripheral processes. Positioning of the electrodes relative to the apical peripheral processes affects the effectiveness and efficiency of the delivered stimulation (e.g., greater separations between electrodes and the targets leads to greater spread of excitation, etc.).

The apical cochlea electrode assembly 150 is inserted into cochlea 140 via an apical cochleostomy 156. As used herein, a cochleostomy is a surgically formed opening formed in the outer wall 142 (FIG. 1A) of cochlea 140 proximate to (at) the apical region 138.

Apical cochlea electrode assemblies in accordance with embodiments presented herein, such as apical cochlea electrode assembly 150, are specifically configured (e.g., sized and dimensioned) so as to be positioned in the apical region of a recipient's cochlea (e.g., beyond an angular position of 720 degrees, which corresponds to final half turn of the cochlea). As a result, the apical cochlea electrode assemblies have different physical or structural characteristics/attributes than traditional basilar electrode assemblies. These different structural characteristics include, for example, smaller size (e.g., smaller cross-sectional area), different shapes, different flexibility, among others, that won't damage the apical structures of the cochlea.

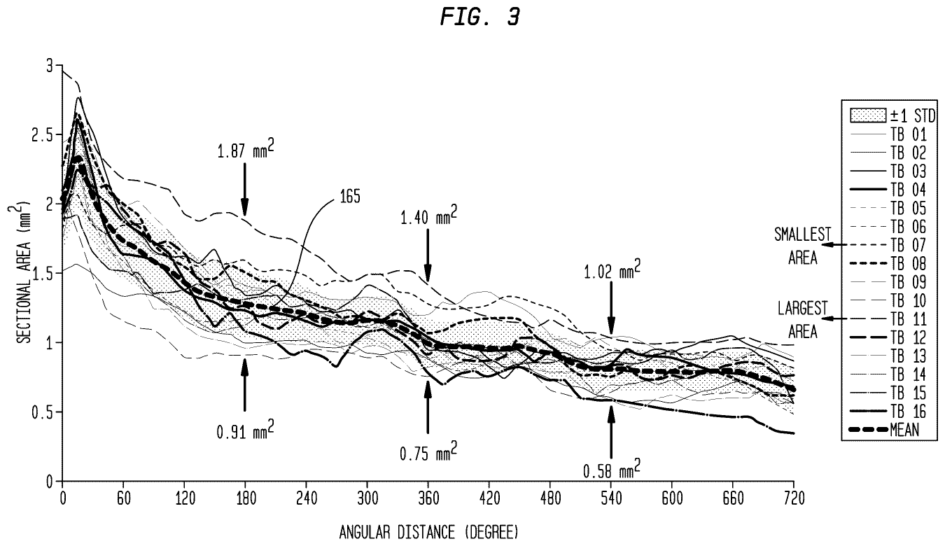

Apical cochlea electrode assemblies in accordance with embodiments presented herein have these different structural characteristics because of the specific structure of the apical region of a cochlea. FIG. 3 is a graph illustrating how the cross-sectional areas of a number of example cochleas change along the length thereof. In particular, FIG. 3 includes a horizontal (x) axis illustrating the angular distance, in degrees, from the basal end of the cochlea, and a vertical (y) axis illustrating the cross-sectional area of the cochlea, square millimeters (mm²). FIG. 3 also includes a line 165 representing a mean of cross-sectional measurements made for a plurality of example cochleas. In general line 165 illustrates that, on average, the cross-sectional areas for the plurality of example cochleas at 720 degrees (i.e., final half turn of the cochlea) decreases to only roughly 50% of the cross-sectional area at 180 degrees. Table 2, below, provides numerical values for the mean cross-sectional areas at different angular distances, as well as the percentage of the mean cross-sectional area relative to the cross-sectional area at 180 degrees, at different angular distances.

| | | Percentage of Mean Cross-sectional |
|---|---|---|
| Angular | Mean Cross-sectional | area relative to Cross-sectional Area at |
| Distance | Area (mm²) | 180 Degrees |
| 180 | 1.28 | 100% |
| 360 | 0.98 | 77% |
| 540 | 0.79 | 62.27% |
| 720 | 0.66 | 51.68% |

In other words, FIG. 3 and Table 2 illustrate that apical region of a recipient's cochlea is significantly different, at least in terms of size (e.g., cross-sectional area), from the basal region of the cochlea. As a result, in to enable insertion of the apical electrode assemblies into the apical region cochlea without damaging the cochlea structures, the smaller size of the apical region of the cochlea requires the apical electrode assemblies to be structurally different (e.g., in terms of size, shape, and flexibility) than traditional electrode assemblies inserted into other portions of a recipient's cochlea. For example, apical cochlea electrode assemblies in accordance with embodiments presented herein have a cross-sectional area that is significantly smaller than a basilar electrode array (e.g., an apical electrode assembly may have an average cross-sectional area that is approximately less than 50% of the average cross-sectional area of a basilar electrode array). Accordingly, traditional electrode assemblies inserted into other portions of a recipient's cochlea are not configured for insertion into the apical region of a recipient's cochlea (e.g., the traditional electrode assemblies would physically not fit into the apical region, would damage the apical region if insertion therein was attempted, etc.).

Additionally, the apex is approximately 900 degrees and, for certain recipients, about 35 mm, away from the basal end of the cochlea 140. Generally speaking, 35 mm is the average distance along the basilar membrane and the distance at the outer wall is even greater Moreover, the cochlea 140 itself is a narrowing, curving tube that has abrupt changes in the vertical rise along its length surrounded by fragile tissue (i.e., the cochlea has turns, but it also has jumps up and down and these differ from recipient to recipient).

Furthermore, due to the closed nature of the cochlea 140, conventional cochlear implant insertions are performed “blind,” meaning the surgeon cannot actually see the electrode assembly as it is inserted into the cochlea and the surgeon relies on touch/feel and experience to properly place the electrode assembly. The tapering physical structure of the apical region of the cochlea, as described above, only increases the difficulty. As such, for these and other reasons, it is a nearly impossible challenge to reach the cochlea apex with a basally inserted electrode assembly without damaging the cochlea 140 itself (i.e., there is a long and challenging path from the cochlea base to the cochlea apex). Therefore, conventional techniques lack the ability to directly stimulate the apical peripheral processes.

In accordance with the techniques presented herein, a specifically configured apical electrode assembly is inserted directly into the apical region of the cochlea. As such, the apical electrodes are positioned in close proximity to the apical peripheral processes and can deliver electrical stimulation directly thereto. Therefore, the techniques presented herein provide the ability to directly stimulate the apical peripheral processes without the challenges associated with insertion of an electrode array from the cochlea base to the cochlea apex.

Returning to FIG. 2B, shown also is the basilar electrode assembly 158 implanted in and/or through the basal region of a recipient's cochlea 140. In the specific embodiment of FIGS. 2A-2C, the basilar electrode assembly 158 comprises a carrier member 160 and twenty-two (22) electrodes 162, sometimes referred to individually as electrodes 162(1)-162(22). The electrodes 162(1)-162(22) are electrically connected to the stimulator unit 147 via one or more wires (not shown in FIGS. 2A-2C) extending through a lead 157. As a result, the electrodes 162(1)-162(22) represent at least twenty-two different stimulation channels. It is to be appreciated that this specific embodiment with twenty-two electrodes is merely illustrative and that the techniques presented herein may be used with one other numbers of electrodes implanted into a recipient's cochlea.

As noted above, the positioning of the apical electrodes 154(1)-154(5) within the apical region 138 of the cochlea 140 enables direct stimulation of the low frequency (apical) peripheral processes (e.g., nerve fibers below approximately 1 kHz) in the apical region 138. As noted above, positioning of the apical electrodes 154(1)-154(5) relative to these target peripheral processes affects the effectiveness and efficiency of the delivered stimulation (e.g., greater separations between electrodes and the target nerve fibers leads to greater spread of excitation, etc.). In contrast, the electrodes 162(1)-162(22) of the basilar electrode assembly 158 are positioned to directly stimulate the high frequency nerve fibers of the cochlea 140 (e.g., nerve fibers above approximately 2 kHz). As such, the stimulation delivered by the electrodes 162(1)-162(22) is sometimes referred to herein as “direct” high frequency stimulation (direct high frequency stimulation channels or high frequency electrodes) because the electrodes 162(1)-162(22) are positioned in close proximity to these target high frequency cells.

The basilar electrode assembly 158 is shown inserted into cochlea 140 via a basal cochleostomy 164. However, it is to be appreciated that the basilar electrode assembly 158 could also be inserted through the round window 161 or the oval window 163.

Also shown in FIG. 2B is an extra-cochlear electrode (ECE) 153 configured to be implanted within the recipient outside of the recipient's cochlea 140. In this example, the extra-cochlear electrode 153 connected to the stimulator unit 147 via one or more wires (not shown in FIGS. 2A-2C) extending through a lead 159.

The stimulator unit 147 includes stimulation circuitry 155 that is configured to generate stimulation (current) signals for delivery to the recipient via, for example, one or more of the apical electrodes 154(1)-154(5), electrodes 162(1)-162(22), etc. As described further below, the stimulation signals electrically stimulate the recipient's auditory nerve fibers in a manner that causes the recipient to perceive captured/received audio signals. Although not shown in FIG. 2, the stimulator unit 147 may also include recording circuitry that is configured to perform electrical measurements via electrodes implanted in, or in proximity to, the cochlea 140, such as via apical electrodes 154(1)-154(5), electrodes 162(1)-162(22), and extra-cochlear electrode 153.

As noted, the cochlear implant 100 includes the external coil 107 and the implantable coil 145. The coils 107 and 145 are typically wire antenna coils each comprised of multiple turns of electrically insulated single-strand or multi-strand platinum or gold wire. Generally, a magnet is fixed relative to each of the external coil 107 and the implantable coil 145. The magnets fixed relative to the external coil 107 and the implantable coil 145 facilitate the operational alignment of the external coil with the implantable coil. This operational alignment of the coils 107 and 145 enables the external component 101 to transmit data, as well as possibly power, to the implantable component 103 via a closely-coupled wireless link formed between the external coil 107 with the implantable coil 145. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 2B illustrates only one example arrangement.

As noted above, the processing block 149 includes sound processing module 151. The sound processing module 151 (e.g., one or more processing elements implementing firmware, software, etc.) is configured to, in general, convert input sound signals into stimulation control signals 137 for use in stimulating a first car of a recipient (i.e., the sound processing module 151 is configured to perform sound processing on input sound signals received at the one or more input devices 133 to generate signals 137 that represent electrical stimulation for delivery to the recipient). The input sound signals that are processed and converted into stimulation control signals may be audio signals received via the microphones 111 or any of the other input devices 133.

In the embodiment of FIG. 2B, the stimulation control signals 137 are provided to the RF transceiver 131, which transcutaneously transfers the stimulation control signals 137 (e.g., in an encoded manner) to the implantable component 103 via external coil 107 and implantable coil 145. That is, the stimulation control signals 137 are received at the RF interface circuitry 127 via implantable coil 145 and provided to the stimulator unit 144. The stimulator unit 144 is configured to utilize the stimulation control signals 137 to generate electrical stimulation signals (e.g., current signals) which, as described further below, are delivered to the recipient's cochlea via the apical electrode assembly 150 and/or the basilar electrode assembly 158. In this way, cochlear implant 100 electrically stimulates the recipient's auditory nerve fibers, bypassing absent or defective hair cells that normally transduce acoustic vibrations into neural activity, in a manner that causes the recipient to perceive one or more components of the input audio signals.

FIGS. 2A and 2B generally illustrate an arrangement in which external component 101 comprises a sound processing unit 113 and a separate external coil 107. In this example, the sound processing unit 113 is a behind-the-ear (BTE) sound processing unit. However, it is to be appreciated that this arrangement is merely illustrate and that embodiments presented herein may be implemented with other external component arrangements. For example, in one alternative embodiment, the external component 101 may comprise an off-the-ear (OTE) sound processing unit in which the external coil, microphones, and other elements are integrated into a single housing/unit configured to worn on the head of the recipient.

It is also to be appreciated that FIGS. 2A and 2B illustrate an arrangement in which the cochlear implant 100 includes an external component. However, it is to be appreciated that embodiments of the present invention may be implemented in cochlear implants having alternative arrangements. For example, elements of the sound processing unit 113 (e.g., such as the processing block 149, power source, etc.), may be implanted in the recipient.

It would be further appreciated that the individual components referenced herein, e.g., microphones 111, auxiliary inputs 129, processing block 149, etc., may be distributed across more than one prosthesis, e.g., two cochlear implants 100, and indeed across more than one type of device, e.g., cochlear implant 100 and a consumer electronic device or a remote control of the cochlear implant 100.

Cochlear implants have been used successfully for many years to treat sensorineural hearing loss. Traditionally, a basilar electrode assembly is inserted into a recipient's cochlea via an opening in the basal region of the cochlea and extends some distance into the cochlea therefrom. Different lengths of basilar electrode assemblies have been proposed and implanted in recipients, thus the insertion distance of basilar electrode assemblies can vary. However, as noted above, due at least in part of the conical spiral structure of the cochlea (i.e., the spiral-like shape) and the delicate anatomical structure of the cochlea, basilar electrode assemblies have a maximum insertion distance in which the most distal electrodes are well short of the apical region of the cochlea. As a result, basilar electrode assemblies only directly stimulate higher frequency tonotopic regions of the cochlea (e.g., higher frequency auditory nerve fibers). For example, certain studies have shown that even the most apical electrodes in a basilar electrode assembly standard electrode arrays mostly stimulate auditory nerve fibers with best frequencies above 2 kHz.

However, the tonotopic regions of the cochlea response to low frequencies, such as frequencies below 1 kHz are believed to have the best temporal precision. As such, this low frequency region would be expected to represent information in difficult listening situations, such as speech in noise, music, etc. and may capture binaural timing cues better than higher frequency regions. Lack of access to these low frequency regions in traditional cochlear implants may contribute to common problems with traditional cochlear implants, such as difficulty with speech in noise, music perception, and binaural timing perception, and frequency-shifted perception of sounds.

The arrangement shown in FIGS. 2A-2C illustrates an enhancement to traditional cochlear implants in that the apical electrodes 154(1)-154(5) provide the ability to directly stimulate the apical region 138 of the cochlea (e.g., the low frequency peripheral processes) and, accordingly, directly activate the peripheral process of the cochlea 140 responsive to low frequencies (e.g., peripheral process associated with frequencies below 1 kHz). In the arrangement of FIGS. 2A-2C, the stimulation provided by apical electrodes 154(1)-154(5) can be combined with and complement stimulation from the electrodes 162(1)-162(22), at the tonotopic regions of the cochlea 140 responsive to high frequencies.

In particular, as described elsewhere herein, the direct stimulation of the apical region 138 of the cochlea 140 via the apical electrodes 154(1)-154(5) is used in coordination with stimulation delivered via the electrodes 162(1)-162(22). That is, the apical cochlear implant 100 (e.g., sound processing module 151) is configured to execute/implement a “full-spectrum coordinated stimulation strategy” to directly stimulate both the high frequency regions of the cochlea 140 (e.g., regions tonotopically mapped to frequencies above 1 kHz), as well as the low frequency regions the cochlea 140 (e.g., regions tonotopically mapped to frequencies below 1 kHz), in order to optimally evoke perception of received sound signals. The apical cochlear implant 100 is referred to as executing a “full-spectrum coordinated stimulation strategy” because the cochlear implant can directly stimulation the high frequency region of the cochlea 140, the low frequency region of the cochlea 140, the ultra-low frequency region of the cochlea 140, and potentially directly or indirectly stimulate the mid frequency region of the cochlea 140.

A cochlear implant in accordance with embodiments presented herein that is configured to execute a full-spectrum coordinated stimulation strategy, which includes direct stimulation of the apical peripheral processes of a recipient's cochlea, may provide a number of benefits over cochlear implants that only directly stimulate the high frequency regions with traditional basilar electrode assemblies alone. These benefits may include, for example, better initial speech understanding, no or reduced perceived frequency shifting, etc. It is also expected that direct stimulation of the apical peripheral processes of the cochlea the will have a significant impact on recipient's suffering from single-sided deafness, for which the frequency shift (frequency offset) is less likely to be overcome given the unchanging acoustic reference at the same tonotopic place in the contralateral car. These and other benefits would accrue from access to low frequency peripheral processes associated with better temporal coding. As such, direct stimulation of these low frequency peripheral processes is expected to aid coding of speech in noise and music. In addition, low frequency pathways are known to be significant in the processing of binaural interaural time difference cues.

Due to the tonotopic mapping of the cochlea 140, different portions of received sound signals are delivered as stimulation signals to different target locations/places of the cochlea 140 via different stimulation channels. As used herein, a stimulation channel is formed by one, or a plurality of electrodes, that are used at a given time instance to deliver stimulation signals (current) to the cochlea 140 so as to elicit stimulation at a specific target location/place of the cochlea. Therefore, apical cochlear implant 100 of FIGS. 2A-2C includes low frequency (apical) stimulation channels provided by the apical electrodes 154(1)-154(5), as well as and high frequency (basal) stimulation channels provided by the electrodes 162(1)-162(22). A stimulation channel may be formed by a single electrode or a plurality of electrodes that operate together to deliver stimulation to a recipient. As such, the apical electrodes 154(1)-154(5) and the electrodes 162(1)-162(22) may be used together to form different numbers of stimulation channels.

In accordance with embodiments presented herein, a full-spectrum coordinated stimulation strategy may be executed in a number of different manners to exploit the different stimulation channels in a manner that optimizes speech understanding for a particular recipient. Provided below are further details regarding embodiments of a full-spectrum coordinated stimulation strategy that may be executed in accordance with examples presented herein.

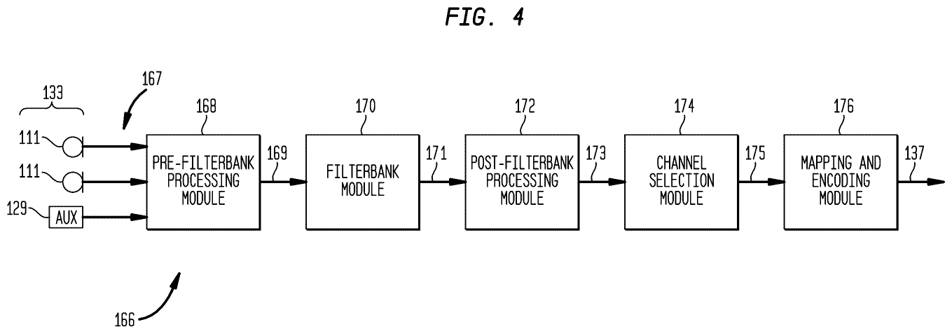

Referring first to FIG. 4, shown is a functional block diagram of a signal processing path 166 of an apical cochlear implant, such as apical cochlear implant 100, in accordance with embodiments presented herein. As noted, the apical cochlear implant 100 comprises one or more input devices 133. In the example of FIG. 4, the one or more input devices comprise two microphones 111 and at least one auxiliary input 119 (e.g., an audio input port, a cable port, a telecoil, etc.). If not already in an electrical form, sound input devices 133 convert received/input sound signals into electrical signals 167, referred to herein as electrical input signals, that represent the received sound signals. As shown in FIG. 4, the electrical input signals 167 are provided to a pre-filterbank processing module 168.

The pre-filterbank processing module 168 is configured to, as needed, combine the electrical input signals 167 received from the sound input devices 133 and prepare those signals for subsequent processing. The pre-filterbank processing module 168 then generates a pre-filtered output signal 169 that, as described further below, is the basis of further processing operations. The pre-filtered output signal 169 represents the collective sound signals received at the sound input devices 133 at a given point in time.

The apical cochlear implant 100 is generally configured to execute sound processing and coding to convert the pre-filtered output signal 169 into stimulation control signals 137 that represent electrical stimulation for delivery to the recipient via the apical electrode assembly 150 and/or the basilar electrode assembly 158. As such, the sound processing path 166 comprises a filterbank module (filterbank) 170, a post-filterbank processing module 172, a channel selection module 174, and a channel mapping and encoding module 176.

In operation, the pre-filtered output signal 169 generated by the pre-filterbank processing module 168 is provided to the filterbank module 170. The filterbank module 170 generates a suitable set of bandwidth limited channels, or frequency bins, that each includes a spectral component of the received sound signals. That is, the filterbank module 170 comprises a plurality of band-pass filters that separate (band-pass filter) the pre-filtered output signal 169 into multiple components/channels (bandwidth limited channels), each one carrying a single frequency sub-band of the original signal (i.e., frequency components of the received sounds signal).

The channels created by the filterbank module 170 are sometimes referred to herein as sound processing channels, and the sound signal components within each of the sound processing channels are sometimes referred to herein as band-pass filtered signals or channelized signals. The band-pass filtered or channelized signals created by the filterbank module 170 are processed (e.g., modified/adjusted) as they pass through the sound processing path 166. As such, the band-pass filtered or channelized signals are referred to differently at different stages of the sound processing path 166. However, it will be appreciated that reference herein to a band-pass filtered signal or a channelized signal may refer to the spectral component of the received sound signals at any point within the sound processing path 166 (e.g., pre-processed, processed, selected, etc.).

As noted above, a feature of the apical cochlear implant 100 is that the stimulation signals may be delivered via the apical electrodes 154(1)-154(5) and/or via the electrodes 162(1)-162(22). The presence of the apical electrodes 154(1)-154(5), which can directly stimulate the apical region 138, may introduce changes to a band-pass filtering process relative to cochlear implants that only utilize a traditional basilar electrode assembly.

For example, the filterbank module 170 may include band-pass filters corresponding to the low frequencies (e.g., below 1 kHz) that have varying spectral widths, and/or spectral widths that are different from (i.e., spectrally narrower) than the band-pass filters corresponding to the band-pass filters corresponding to the high frequencies. In other words, the filterbank module 170 may have band-pass filters (e.g., implemented as part of a fast Fourier transform (FFT)) having different or variable spectral widths such that the resulting bandwidth limited channels have different or variable spectral widths. For example, the spectral widths of the bandwidth limited channels associated with low frequencies of the cochlea may be narrower than the bandwidth limited channels associated with high frequencies of the cochlea (e.g., bandwidth limited channels corresponding to low frequencies have spectral widths that are narrower than bandwidth limited channels corresponding to high frequencies of the sound signals). In certain embodiments, the spectral width of a band-pass filter of the filterbank module 170 may depend on the associated tonotopic location of the cochlea 140 (e.g., bandwidth limited channels corresponding to low frequencies have spectral widths that are narrower than bandwidth limited channels corresponding to high frequencies of the sound signals). Therefore, the filterbank module 170 is configured to band-pass filter the received sound signals with a plurality of band-pass filters to generate a set of bandwidth limited channels each including a spectral component of the received sound signals. In certain embodiments, the plurality of band-pass filters have a non-uniform spectral width.

The arrangement of FIG. 4 that uses spectrally narrower band-pass filters for the low frequencies may facilitate the delivery of more acoustic information/detail at the apical region 138 where it is most critical for speech/sound perception. Similarly, the use of spectrally wider band-pass filters for the high frequencies enables the cochlear implant 100 to deliver less acoustic detail in the other regions of the cochlea 140, where the acoustic detail may not be as needed and/or as critical for sound perception.

Returning to FIG. 4, at the output of the filterbank module 170, the channelized signals are initially referred to herein as pre-processed channelized signals 171. The number ‘m’ of channels and pre-processed channelized signals 171 generated by the filterbank module 170 may depend on a number of different factors including, but not limited to, implant design, number of active electrodes, coding strategy, and/or recipient preference(s).

The pre-processed channelized signals 171 are provided to the post-filterbank processing module 172. The post-filterbank processing module 172 is configured to perform a number of sound processing operations on the pre-processed channelized signals 171. These sound processing operations include, for example, channelized gain adjustments for hearing loss compensation (e.g., gain adjustments to one or more discrete frequency ranges of the sound signals), noise reduction operations, speech enhancement operations, etc., in one or more of the channels. After performing the sound processing operations, the post-filterbank processing module 172 outputs a plurality of processed channelized signals 173.

In the specific arrangement of FIG. 4, the sound processing path 166 includes a channel selection module 174. The channel selection module 174 is configured to perform a channel selection process to select, according to one or more selection rules, which of the ‘m’ channels are to be use in hearing compensation. The signals selected at channel selection module 174 are represented in FIG. 4 by arrow 175 and are referred to herein as selected channelized signals or, more simply, selected signals.

In the embodiment of FIG. 4, the channel selection module 174 selects a subset ‘n’ of the ‘m’ processed channelized signals 173 for use in generation of electrical stimulation for delivery to a recipient (i.e., the sound processing channels are reduced from ‘m’ channels to ‘n’ channels). In one specific example, the ‘n’ largest amplitude channels (maxima) from the ‘m’ available combined channel signals/masker signals is made, with ‘m’ and ‘n’ being programmable during initial fitting, and/or operation of the prosthesis. It is to be appreciated that different channel selection methods could be used, and are not limited to maxima selection.

It is also to be appreciated that, in certain embodiments, the channel selection module 174 may be omitted. For example, certain arrangements may use a continuous interleaved sampling (CIS), CIS-based, hybrid of channel selection and F0 derived coding, or other non-channel selection sound coding strategy.

The sound processing path 166 also comprises the channel mapping module 176. The channel mapping module 176 is configured to map the amplitudes of the selected signals 175 (or the processed channelized signals 173 in embodiments that do not include channel selection) into a set of stimulation control signals (e.g., stimulation commands) 137 that represent the attributes of the electrical stimulation signals that are to be delivered to the recipient so as to evoke perception of at least a portion of the received sound signals. This channel mapping may include, for example, threshold and comfort level mapping, dynamic range adjustments (e.g., compression), volume adjustments, etc., and may encompass selection of various sequential and/or simultaneous stimulation strategies.

In the embodiment of FIG. 4, the set of stimulation commands that represent the electrical stimulation signals are encoded for transcutaneous transmission (e.g., via an RF link) to an implantable component 104 (FIGS. 1A and 1B). This encoding is performed, in the specific example of FIG. 4, at the channel mapping module 176. As such, channel mapping module 176 is sometimes referred to herein as a channel mapping and encoding module and operates as an output block configured to convert the plurality of channelized signals into the plurality of stimulation control signals 137.

As noted above, an apical cochlear implant 100 executing a full-spectrum coordinated stimulation strategy in accordance with embodiments presented herein may leverage the direct access to the peripheral processes, particularly in the apical region 138, through the use of different spectral width band-filters at the filterbank module 170. In certain embodiments, the apical cochlear implant 100 may also or alternatively leverage the direct access to the peripheral processes of the apical region 138 through physical electrode spacing in the apical electrode assembly 150 that is different from that in the basilar electrode assembly 158 (e.g., the electrodes 154(1)-154(5) have a spacing that is smaller than that of the electrodes 162(1)-162(26).

Additionally or alternatively, the apical cochlear implant 100 may leverage the direct access to the peripheral processes in the apical region 138 through use of various stimulation resolutions (e.g., use of different electrode configurations).

Electrical stimulation of nerve cells operates by causing selected groups of nerve calls to fire/activate. In order for a nerve cell to fire, the nerve cell must first obtain a membrane voltage above a critical threshold. The number of nerve cells that fire in response to electrical stimulation can affect the “resolution” of the electrical stimulation. As used herein, the resolution of the electrical stimulation or the “stimulus resolution” refers to the amount of acoustic detail (i.e., the spectral and/or temporal detail from the input acoustic sound signal(s)) that is delivered by the electrical stimulation at the implanted electrodes in the cochlea and, in turn, received by the primary auditory neurons (spiral ganglion cells). As described further below, electrical stimulation has a number of characteristics/attributes that control the stimulus resolution. These attributes include for example, the spatial attributes of the electrical stimulation, temporal attributes of the electrical stimulation, instantaneous spectral bandwidth attributes of the electrical stimulation, etc.

The spatial attributes of the electrical stimulation control the width along the frequency axis (i.e., along the basilar membrane) of an area of activated nerve cells in response to delivered stimulation, sometimes referred to herein as the “spatial resolution” of the electrical stimulation. The temporal attributes refer to the temporal coding of the electrical stimulation, such as the pulse rate, sometimes referred to herein as the “temporal resolution” of the electrical stimulation. The instantaneous spectral bandwidth attributes refer to the proportion of the analyzed spectrum that is delivered via electrical stimulation, such as the number of channels stimulated out of the total number of channels in each stimulation frame.

The spatial resolution of electrical stimulation may be controlled, for example, through the use of different electrode configurations for a given stimulation channel to activate nerve cell regions of different widths. Monopolar stimulation, for instance, is an electrode configuration where for a given stimulation channel the current is “sourced” via one of the electrodes within the cochlea, but the current is “sunk” by a far-field electrode, such as the extra-cochlear electrode 153 (FIG. 1A). Monopolar stimulation typically exhibits a large degree of current spread (i.e., wide stimulation pattern) and, accordingly, has a low spatial resolution. Other types of electrode configurations, such as bipolar, tripolar, focused multi-polar (FMP), a.k.a. “phased-array” stimulation, etc. typically reduce the size of an excited neural population by “sourcing” the current via one or more electrodes within the cochlea, while also “sinking” the current via one or more other electrodes located near (proximate to) the current sourcing electrodes. Bipolar, tripolar, focused multi-polar and other types of electrode configurations that both source and sink current via electrodes are generally and collectively referred to herein as “focused” stimulation. Focused stimulation typically exhibits a smaller degree of current spread (i.e., narrow stimulation pattern) when compared to monopolar stimulation and, accordingly, has a higher spatial resolution than monopolar stimulation. Likewise, other types of electrode configurations, such as double electrode mode, virtual channels, wide channels, defocused multi-polar, etc. typically increase the size of an excited neural population by “sourcing” the current via multiple neighboring electrodes.

Again, as noted, apical cochlear implant 100 may leverage the direct access to the peripheral processes in the apical region 138 through the use of various stimulus resolutions. For example, in certain embodiments presented herein, apical cochlear 100 may use focused stimulation within the apical region 138 such that stimulation signals delivered via the apical electrodes 154(1)-154(5) stimulate only a narrow region of neurons such that the resulting neural responses from neighboring stimulation channels have minimal overlap. Such a strategy may better mimic natural hearing and enable better perception of the details of the sound signals (i.e., greater control of current flow to create discriminable pitches).

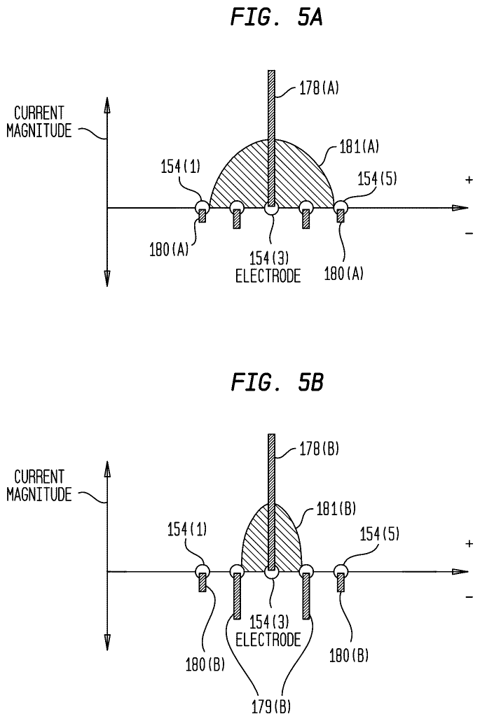

FIGS. 5A and 5B illustrate focused channel configurations where intracochlear compensation currents are added to decrease the spread of current along the frequency axis of the cochlea (i.e., one or more of the apical electrodes source/deliver current and one or more of the apical electrodes return/sink current). The compensation currents are delivered with a polarity that is opposite to that of a primary/main current. In general the more compensation current at nearby electrodes, the more focused the resulting stimulation pattern (i.e., the lower the width of the stimulus patterns increase and thus increasingly higher spatial resolutions). That is, the spatial resolution is increased by introducing increasing large compensation currents on electrodes surrounding the central electrode with the positive current.

More specifically, in FIG. 5A positive stimulation current 178(A) is delivered via electrode 154(3) and stimulation current 180(A) of opposite polarity is delivered via the neighboring electrodes, namely electrodes 154(1), 154(2), 154(4), and 154(5). The intra-cochlear stimulation currents 178(A) and 180(A) generate a stimulation pattern 181(A) which, as shown, only spreads across electrodes 154(2)-154(4)). In FIG. 5B, positive stimulation current 178(B) is delivered via electrode 154(B), while stimulation current 179(B) of opposite polarity is delivered via the neighboring electrodes 154(2) and 154(4), and second stimulation current 180(B) also of opposite polarity is delivered via the electrodes 154(1) and 154(5) The stimulation currents 178(B), 179(B), and 180(B) generate a stimulation pattern 181(B) which, as shown, is generally localized to the spatial area adjacent electrode 154(3).

The difference in the stimulation patterns 181(A) and 181(B) in FIGS. 5A and 5B, respectively, is due to the magnitudes (i.e., weighting) of opposite polarity current delivered via the neighboring electrodes 154(1), 154(2), 154(3), and 154(4). In particular, FIG. 5A illustrates a partially focused configuration where the compensation currents do not fully cancel out the main current on the central electrode and the remaining current goes to a far-field extracochlear electrode (not shown). FIG. 5B is a fully focused configuration where the compensation currents fully cancel out the main current on the central electrode 154(4) (i.e., no far-field extracochlear electrode is needed). It is to be appreciated that the two focused stimulation patterns shown in FIGS. 5A and 5B are merely illustrative and that, as noted above, focused stimulation delivered via the apical electrodes 154(1)-154(5) may take a number of different arrangements.

In accordance with embodiments presented herein, the use of focused stimulation in the apical region 138 (e.g., delivered via the apical electrodes 154(1)-154(5)) may be combined with the delivery of focused stimulation to the high frequency regions of the cochlea 140 (e.g., via electrodes 162(1)-162(22)). Alternatively, the use of focused stimulation in the apical region 138 (e.g., delivered via apical electrodes 154(1)-154(5)) may be combined with the delivery of de-focused or monopolar stimulation to the high frequency regions of the cochlea 140 (e.g., via electrodes 162(1)-162(22)). Therefore, in accordance with embodiments presented herein, the apical cochlear implant 100 may be configured to employ different electrode configurations at each of the apical electrode assembly 150 and the basilar electrode assembly 158 (e.g., one electrode configuration for the direct low frequency stimulation channels and a different electrode configuration for the direct high frequency stimulation channels).

It is to be appreciated that the apical electrodes 154(1)-154(5) may use non-focused electrode configurations in addition to, or as an alternative to, focused stimulation and for current steering. For example, in certain embodiments, the apical electrodes 154(1)-154(5) could each operate as independent stimulation channels to stimulate portions of the apical region 138. In these embodiments, the extra-cochlear electrode 153 and/or one or more of the electrodes 162(1)-162(22) may function as the current return electrode.

As noted, increased spatial resolution is one technique that may be employed by the apical cochlear implant 100 for delivery of stimulation signals to the apical region 138. In further embodiments, the apical cochlear implant 100 may utilize different temporal resolutions (e.g., different pulse rates) at each of the apical electrode assembly 150 and the basilar electrode assembly 158 (e.g., one pulse rate for the direct low frequency stimulation channels and a different pulse rate for the direct high frequency stimulation channels).

In traditional cochlear implants with only a basilar electrode assembly, monopolar stimulation includes the delivery of current signals via one of the electrodes. The current is then sunk/returned via an extra-cochlear electrode. In accordance with certain embodiments presented herein, the apical electrodes 154(1)-154(5) may be used as current returns (current sinks) for electrical stimulation delivered via the electrodes 162(1)-162(22) of the basilar electrode assembly 158. The use of the electrodes 154(1)-154(5) as current returns may sharpen the monpolar stimulation via a given electrode 162(1)-162(22).

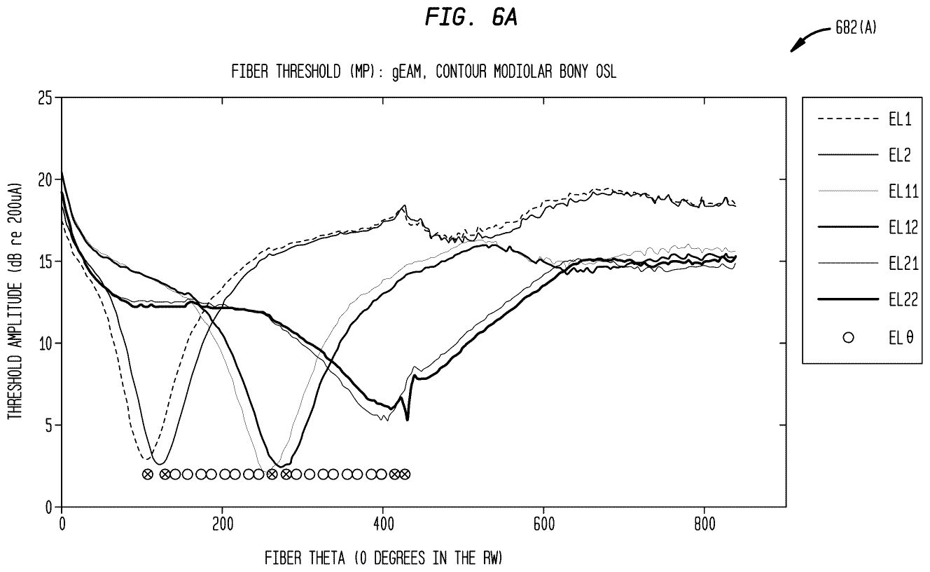

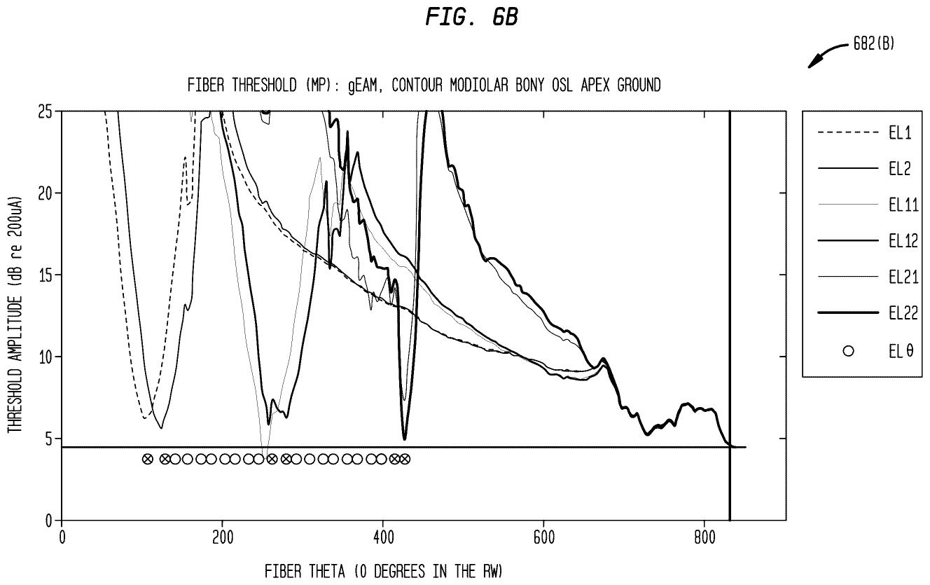

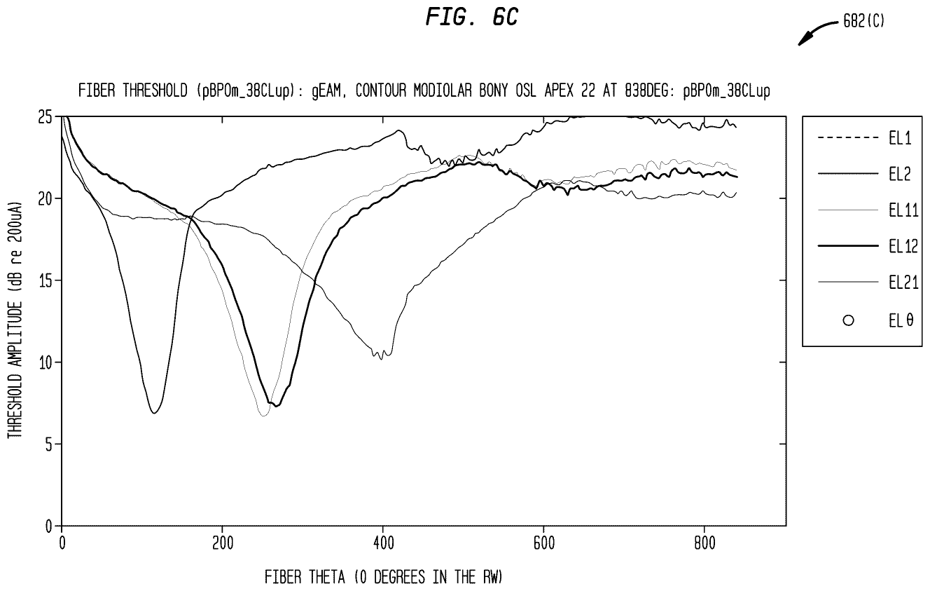

More specifically, FIG. 6A is a graph 682(A) in which the vertical (y) axis illustrates threshold amplitudes (in dB re 2000 μA), while the horizontal (x) axis illustrates the angular cochlea depth, in degrees, where zero degrees corresponds to the round window location. FIG. 6A also includes six (6) traces illustrating the threshold amplitudes for monopolar stimulation delivered via each of a plurality of electrodes of a standard electrode assembly (i.e., where the electrodes are located in the high frequency region of the cochlea). In FIG. 6A, trace EL1 corresponds to the most basal/proximal electrode, while trace EL22 corresponds to the most apical/distal electrode. In FIG. 6A, all of the stimulation current is sunk via an extra-cochlear electrode. As can be seen in FIG. 6A, the sinking of all of the current via the extra-cochlear electrode results in the most apical response is at roughly 440 degrees and the responses are relatively broad (e.g., excitation spread excites a broad degree range of nerve fibers).

FIG. 6B is a graph 682(B) in which, similar to FIG. 6A, the vertical axis illustrates threshold amplitudes, while the horizontal axis illustrates the angular cochlea depth, in degrees. FIG. 6B illustrates an apical cochlear implant arrangement comprising a basilar electrode assembly with a plurality of electrodes positioned in a high frequency portion of the cochlea, and an apical electrode assembly with a plurality of electrodes positioned in the frequency portion of the cochlea (i.e., the portion below 1000 kHz).

FIG. 6B also includes six (6) traces illustrating the threshold amplitudes for biphasic stimulation delivered via the electrodes of the basilar electrode assembly. Again, trace EL1 corresponds to the most basal/proximal electrode, while trace EL22 corresponds to the most apical/distal electrode. In FIG. 6B, all of the stimulation current delivered via the basilar electrode assembly is sunk via one or more of the electrodes of the apical electrode assembly (i.e., electrodes positioned in the low frequency portion of the cochlea).

As can be seen in FIG. 6B, the sinking of the return current in this manner results in much sharper (narrower) results than the monopolar arrangement of FIG. 6A (e.g., excitation spread excites a smaller degree range of nerve fibers than in FIG. 6A), but a large number of the neurons at the apex are activated at 4 dB re 200 μA. FIG. 6B also illustrates an undesirable second stimulation site (e.g., at approximately 750 degrees), sometimes referred to as a bipolar pitch percept.

FIG. 6C is a graph 682(C) in which, similar to FIG. 6A, the vertical axis illustrates threshold amplitudes, while the horizontal axis illustrates the angular cochlea depth, in degrees. FIG. 6C illustrates an apical cochlear implant arrangement comprising a basilar electrode assembly with a plurality of electrodes positioned in a high frequency portion of the cochlea, and an apical electrode assembly with a plurality of electrodes positioned in the frequency portion of the cochlea (i.e., the portion below 1000 kHz).

FIG. 6C also includes six (6) traces illustrating the threshold amplitudes for partial bipolar stimulation delivered via the electrodes of the basilar electrode assembly. Again, trace EL1 corresponds to the most basal/proximal electrode, while trace EL22 corresponds to the most apical/distal electrode. In FIG. 6C, a portion of the stimulation current is sunk via an extra-cochlear electrode, while another portion is sunk by one or more of the electrodes of the apical electrode assembly (i.e., electrodes positioned in the low frequency portion of the cochlea). In particular, the current return is substantially split between the extra-cochlear electrode and the one or more electrodes of the apical electrode assembly (e.g., +1.0 current is delivered via the basilar electrode assembly, the extra-cochlear electrode receives/sinks −0.5 and the one or more electrodes of the apical electrode assembly receive/sink −0.5). As can be seen in FIG. 6C, the splitting of the return current in this manner results in higher thresholds than the monopolar arrangement of FIG. 6A. Additionally, the responses are narrower than those of FIG. 6A (e.g., excitation spread excites a smaller degree range of nerve fibers than in FIG. 6A), but not as narrow as those in FIG. 6B. In other words, FIG. 6C illustrates a sharpened response without creating an undesirable bipolar pitch percept.

Collectively, FIGS. 6A-6C illustrate that the splitting the current return between (e.g., the relative amount of current sunk by) an extra-cochlear electrode and one or more apical electrodes within the apical region may optimize/shape the excitation patterns induced by the basilar stimulation channels. The amount of current that is returned/sunk via each of the extra-cochlear electrode and the one or more apical electrodes may be adjusted, for example, based on recipient-specific attributes during a fitting sessions. The amount of current that is returned/sunk via each of the extra-cochlear electrode and the one or more apical electrodes may also be different for different ones of the electrodes in the basilar electrode assembly and the optimum proportion could be a variable which changes with respect to electrode position. For example, more basilar electrodes may require a larger proportion of current through the apical electrodes of the apical electrode assembly to achieve the desired shift in frequency response. More apical electrodes of the basilar electrode assembly may require a lower proportion of current through the apical electrodes of the apical electrode assembly since they are closer to the apex and therefore more sensitive to current flow there.

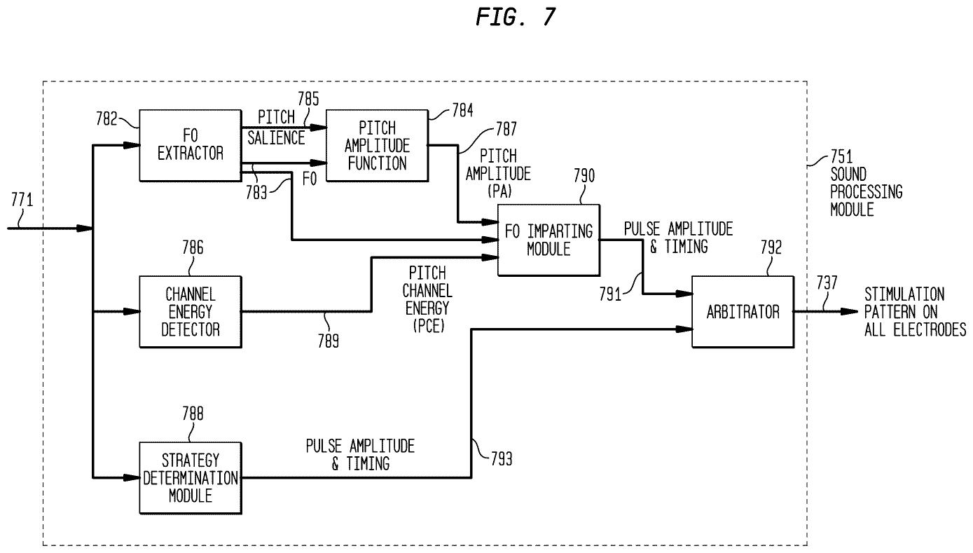

As noted above, a full-spectrum coordinated stimulation strategy in accordance with embodiments presented herein may be executed in a number of different manners. In certain examples, the full-spectrum coordinated stimulation strategy may include specific sound coding techniques that optimize speech/sound understanding through use of an apical electrode assembly. FIGS. 7-12 illustrate further details of example sound coding techniques that exploit the positioning of the apical electrodes to directly stimulate the apical region (i.e., electrically stimulate low frequency peripheral processes via apical electrodes of an apical electrode assembly in conjunction with stimulation via electrodes of a basilar electrode assembly to take advantage of the apical placement and the direct low frequency pathway).

Referring first to FIG. 7, shown is functional block of a sound processing module 751 of an apical cochlear implant to optimize speech/sound understanding through use of an apical electrode assembly. The sound processing module 751 is configured to execute a fundamental frequency (F0) imparting/encoding process to provide enhanced pitch cues within the stimulation signals delivered to the apical peripheral processes of a cochlear implant via apical electrodes of an apical electrode array (i.e., adjust an initial stimulation strategy based on fundamental frequency and channel energy to provide enhanced pitch cues at the apical stimulation channels).

It is to be appreciated that this logical arrangement of FIG. 7 is merely illustrative and that operations represented in FIG. 7 may be performed in a number of different manners and may be split across different functional elements and, in certain examples, across different devices. It is also to be appreciated that the sound processing module 751 may perform other operations which, for case of illustration, have been omitted from FIG. 7.

In the embodiment of FIG. 7, the sound processing module 751 includes, among other elements, a fundamental frequency (F0) extractor 782, a pitch amplitude function 784, a channel energy detector 786, a strategy determination module 788, an F0 imparting module 790, and an arbitrator 792. As described further below, FIG. 7 illustrates an embodiment in which F0 extraction and energy detection are used to adjust an initial/primary stimulation strategy.

More specifically, in the example of FIG. 7, the sound processing module 751 receives pre-processed channelized signals 771 (e.g., similar to pre-processed channelized signals 171 generated by the filterbank module 170 in FIG. 4). The pre-processed channelized signals 771 are provided to the F0 extractor 782, the channel energy detector 786, and the strategy determination module 788. Although FIG. 7 is described with reference to the use of processed channelized signals 771, it also to be appreciated that the operations may be performed, at least in part, using other versions of the received sound signals (e.g., the electrical input signals received from the sound input, the pre-filtered output signals, etc.).

The F0 extractor 782 is configured to extract the fundamental frequency (F0) of the received sound signals (i.e., the signals used to generate the processed channelized signals 771). The outputs of the F0 extractor 782 are the fundamental frequency 783 and a measure 785 of the confidence of the presence of the fundamental frequency. The measure 785, sometimes referred to as the “pitch salience,” provides an indication of the harmonicity of the received sound signals. It is possible to specify a fundamental frequency limit (F0_limit) by characterizing the recipient's pulse following abilities (e.g., psychophysically, electrophysiological, etc.). For example, the fundamental frequency limit may be 500 Hz, although this value is merely illustrative.

The fundamental frequency 783 and the pitch salience 785 are provided to the pitch amplitude function 784. The pitch amplitude function 784 is configured to determine an estimate or likelihood that the fundamental frequency 783 is correct (i.e., determine how tone-like is the signal in the lowest frequency bin). The pitch amplitude function 784 outputs a pitch amplitude 787. In certain examples, the pitch amplitude 787 is a function (based on) the pitch salience (e.g., a step function if the pitch salience crosses some level or sigmoidal function for more gradual application). Alternatively, the pitch amplitude 787 is a function of the pitch salience and the fundamental frequency (e.g., includes multiplier of negative sigmoidal function of F0 such that as F0 rises beyond the F0_limit the pitch amplitude is weaker).

As noted, the pre-processed channelized signals 771 are provided to the channel energy detector 786. The channel energy detector 786 is configured to determine the energy in the lowest frequency channel, independent of whether the signal is harmonic/regular or not, and output a pitch channel energy (PCE) 789 In one example, the channel energy detector 786 extracts the energy in a broad band signal (e.g., the electrical input signals 167 received from the sound input devices 133 or the pre-filtered output signal 169) to drive the apical stimulation channel(s) (i.e., the F0 imparting electrode(s) at the apical peripheral processes). In another example, the channel energy detector 786 extracts energy in only a low frequency range/band (e.g., below 800 Hz). The low frequency range could be adjusted on a per-recipient basis to match tonotopy, e.g., by measuring acoustic pitch in contralateral car related to an apical electrode and next electrode, and determine appropriate frequency break point.

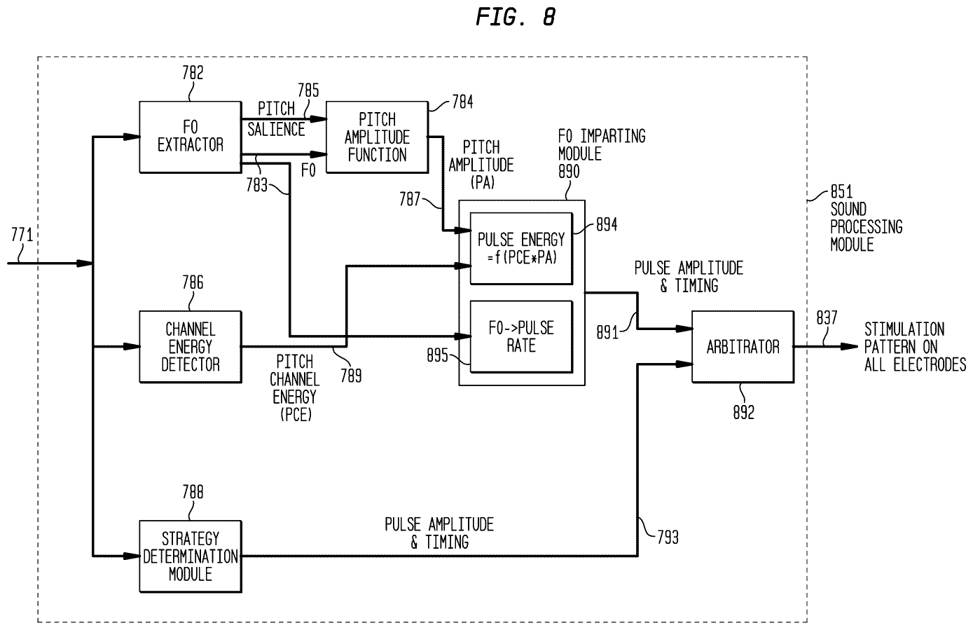

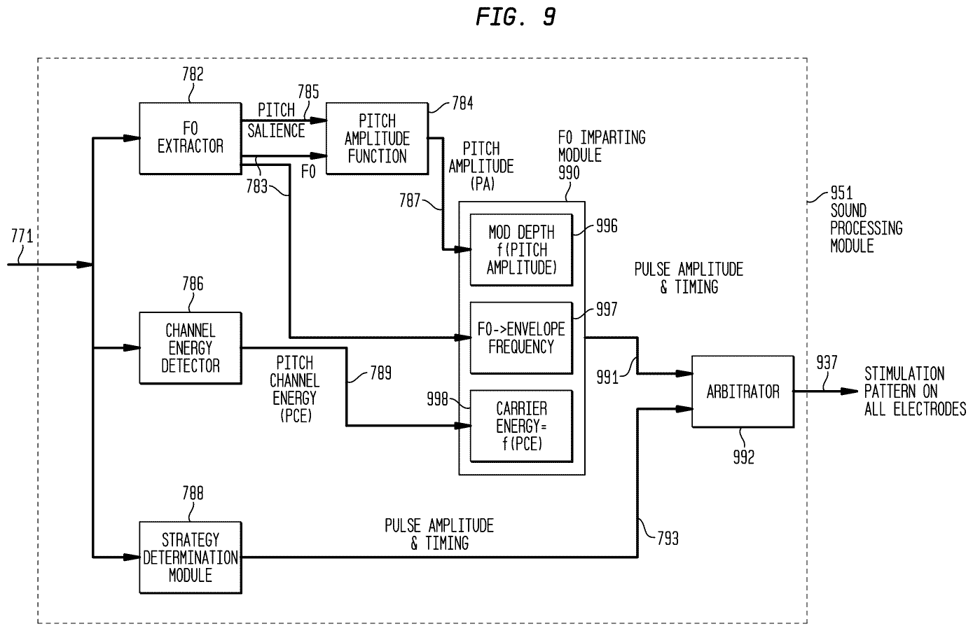

The F0 imparting module 790 receives the pitch amplitude 787, the fundamental frequency 783, and the pitch channel energy 789. The F0 imparting module 790 is configured to analyze these values and determine pulse control parameters 791 (e.g., pulse amplitude and timing) to encode the fundamental frequency. FIGS. 8 and 9 provide further details of two example implementations of the F0 imparting module 790.

The strategy determination module 788 is configured to generate pulse control parameters 793 (e.g., pulse amplitude and amplitude timing) in accordance with an initial pre-determined/pre-set strategy. That is, the strategy determination module 788 uses an initial strategy to determine pulse control parameters 793 that are configured to impart the sound information included in the received sound signals, other than the fundamental frequency information. The initial strategy used to generate the pulse control parameters 793 may be, for example, a continuous interleaved sampling (CIS) strategy, an Advanced Combination Encoder (ACE) strategy, a 500 pps strategy, etc. In certain examples, it may be possible use a lower update rate strategy which will complement the F0 imparting electrode.

The pulse control parameters 791 to encode the fundamental frequency are provided to an arbitrator 792. In addition to these pulse control parameters 791, the arbitrator 792 also receives the pulse control parameters 793 from the strategy determination module 788. The arbitrator 792 analyzes the pulse control parameters 791 and the pulse control parameters 793 to generate stimulation control signals 737 that represent electrical stimulation for delivery to the recipient via an apical electrode assembly and/or a basilar electrode assembly of the cochlear implant. In certain examples, the F0 imparting electrode(s) (e.g., one or more of the apical electrodes implanted in the apical region) will have high weighting in the arbitration decisions. In certain embodiments, a lower amplitude of pulse energy on the lowest frequency (pitch) channel could reduce the arbitration weight, but in general it would excerpt more control relative to other channels. That is, the arbitration decisions may be configured to be faithful to the low frequency timing, thus the arbitration process favors the low frequency channels when it determines the stimulation control signals 737.

FIG. 8 is a functional block diagram illustrating further details of one example implementation of an FO imparting module in accordance with embodiments presented herein. In the example of FIG. 8, pulse rate is used to impart/encode the fundamental frequency in the low frequency stimulation signals.

More specifically, shown in FIG. 8 is a functional block of a sound processing module 851 of an apical cochlear implant to optimize speech/sound understanding through use of an apical electrode assembly. It is to be appreciated that this logical arrangement of FIG. 8 is merely illustrative and that operations represented in FIG. 8 may be performed in a number of different manners and may be split across different functional elements and, in certain examples, across different devices. It is also to be appreciated that the sound processing module 851 may perform other operations which, for case of illustration, have been omitted from FIG. 8.

Similar to the sound processing module 751 of FIG. 7, the sound processing module 851 includes, among other elements, the fundamental frequency (F0) extractor 782, the pitch amplitude function 784, the channel energy detector 786, and the strategy determination module 788, each implemented as described above with reference to FIG. 7. The sound processing module also includes an F0 imparting module 890 and an arbitrator 892.

As noted above, the F0 extractor 782 is configured to extract the fundamental frequency (F0) of the received sound signals and output the fundamental frequency 783 and the pitch salience 785. The fundamental frequency 783 and the pitch salience 785 are provided to the pitch amplitude function 784, which outputs the pitch amplitude 787. As noted, the channel energy detector 786 is configured to determine the energy in the lowest frequency channel, independent of whether the signal is harmonic/regular or not, and output a pitch channel energy (PCE) 789.

The F0 imparting module 890 receives the pitch amplitude 787, the fundamental frequency 783, and the pitch channel energy 789. The F0 imparting module 890 includes a pulse energy block 794 and a pulse rate block 795. In the example of FIG. 8, the F0 imparting module 890 is configured to impart the fundamental frequency via pulse rate. In particular, the fundamental frequency determines the pulse rate and the pulse amplitude is determined by the pitch channel energy 789 and the pitch amplitude 787. Accordingly, the F0 imparting module 890 determines and outputs pulse control parameters 891 (e.g., pulse amplitude and timing) to encode the fundamental frequency. The pulse control parameters 891 are provided to the arbitrator 892.

As noted above, the strategy determination module 788 is configured to generate pulse control parameters 793 (e.g., pulse amplitude and amplitude timing) in accordance with an initial pre-determined/pre-set strategy, and provide this parameters to the arbitrator 892. The arbitrator 892 may operate similar to arbitrator 792 to analyze the pulse control parameters 891 and the pulse control parameters 793 to generate stimulation control signals 837 that represent electrical stimulation for delivery to the recipient via an apical electrode assembly and/or a basilar electrode assembly of the cochlear implant.

It is to be appreciated that, in the example of FIG. 8, phase/timing cues could be restored to the F0 imparting process” by additional signal analysis methods (e.g., zero crossing or peak picking). Such methods could preserve interaural time difference cues.

FIG. 9 is a functional block diagram illustrating further details of one example implementation of an F0 imparting module in accordance with embodiments presented herein. In the example of FIG. 9, envelope modulation is used to impart/encode the fundamental frequency in the low frequency stimulation signals.

More specifically, shown in FIG. 9 is a functional block of a sound processing module 951 of an apical cochlear implant to optimize speech/sound understanding through use of an apical electrode assembly. It is to be appreciated that this logical arrangement of FIG. 9 is merely illustrative and that operations represented in FIG. 9 may be performed in a number of different manners and may be split across different functional elements and, in certain examples, across different devices. It is also to be appreciated that the sound processing module 951 may perform other operations which, for ease of illustration, have been omitted from FIG. 9.

Similar to the sound processing module 751 of FIG. 7, the sound processing module 951 includes, among other elements, the fundamental frequency (F0) extractor 782, the pitch amplitude function 784, the channel energy detector 786, and the strategy determination module 788, each implemented as described above with reference to FIG. 7. The sound processing module also includes an F0 imparting module 990 and an arbitrator 992.

As noted above, the F0 extractor 782 is configured to extract the fundamental frequency (F0) of the received sound signals and output the fundamental frequency 783 and the pitch salience 785. The fundamental frequency 783 and the pitch salience 785 are provided to the pitch amplitude function 784, which outputs the pitch amplitude 787. As noted, the channel energy detector 786 is configured to determine the energy in the lowest frequency channel, independent of whether the signal is harmonic/regular or not, and output a pitch channel energy (PCE) 789.

The F0 imparting module 990 receives the pitch amplitude 787, the fundamental frequency 783, and the pitch channel energy 789. The F0 imparting module 990 includes a modulation depth block 996 configured to determine the modulation depth from the pitch amplitude 787, where greater pitch amplitude results in greater modulation depth. The F0 imparting module 990 also includes an envelope block 997 that determines the modulation rate based on the fundamental frequency 783. Additionally, the F0 imparting module 990 includes a carrier energy block configured to determine the carrier energy based on the pitch channel energy 789. The pulse amplitude is determined based on the modulation rate, pitch channel energy 789, and the pitch amplitude 787. The pulse train rate could be, for example, 1200 pps, 1800 pps, etc. As such, the F0 imparting module 990 imparts the fundamental frequency via envelope modulation of a high rate pulse train.

Accordingly, the F0 imparting module 990 determines and outputs pulse control parameters 991 (e.g., pulse amplitude, modulation depth, rate, etc.) to encode the fundamental frequency. The pulse control parameters 991 are provided to the arbitrator 992.

As noted above, the strategy determination module 788 is configured to generate pulse control parameters 793 (e.g., pulse amplitude and amplitude timing) in accordance with an initial pre-determined/pre-set strategy, and provide this parameters to the arbitrator 992. The arbitrator 992 may operate similar to arbitrator 792 to analyze the pulse control parameters 991 and the pulse control parameters 793 to generate stimulation control signals 937 that represent electrical stimulation for delivery to the recipient via an apical electrode assembly and/or a basilar electrode assembly of the cochlear implant.

It is to be appreciated that, in the example of FIG. 9, phase/timing cues could be restored to the F0 imparting process by additional signal analysis methods (e.g., zero crossing or peak picking). Such methods could preserve interaural time difference cues.

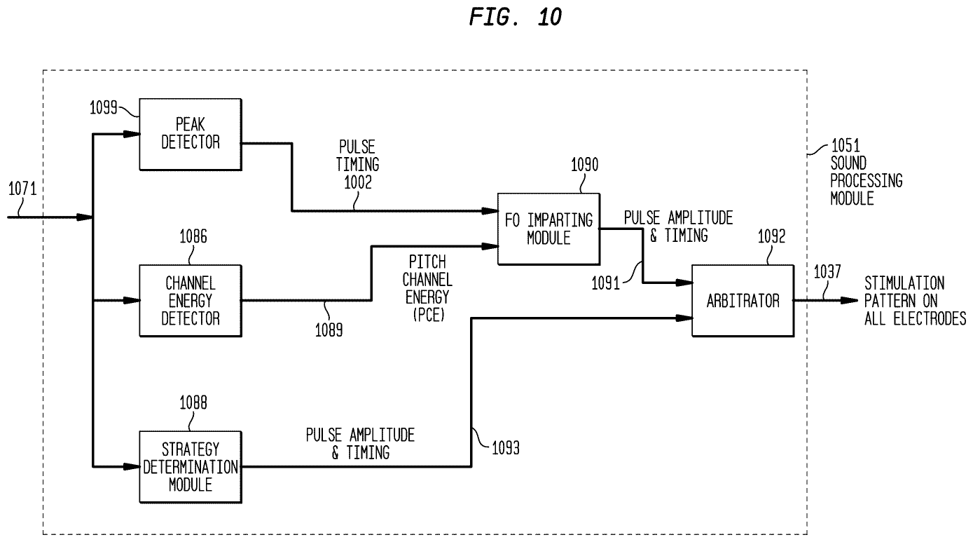

FIG. 10 is functional block of a sound processing module 1051 of an apical cochlear implant to optimize speech/sound understanding through use of an apical electrode assembly. The sound processing module 1051 is configured to execute a fundamental frequency (F0) imparting process to provide enhanced pitch cues within the stimulation signals delivered to the apical peripheral processes via apical electrodes of an apical electrode array (i.e., adjust an initial stimulation strategy based on fundamental frequency and channel energy to provide enhanced pitch cues at the apical stimulation channels). In this example, the fundamental frequency is imparted/encoded via a peak selection (peak picking) process/technique.

It is to be appreciated that the logical arrangement of FIG. 10 is merely illustrative and that operations represented in FIG. 10 may be performed in a number of different manners and may be split across different functional elements and, in certain examples, across different devices. It is also to be appreciated that the sound processing module 1051 may perform other operations which, for case of illustration, have been omitted from FIG. 10.

In the embodiment of FIG. 10, the sound processing module 1051 includes, among other elements, a peak selector 1099, a channel energy detector 1086, a strategy determination module 1088, an F0 imparting module 1090, and an arbitrator 1092. In the example of FIG. 10, the sound processing module 1051 receives pre-processed channelized signals 1071 (e.g., similar to pre-processed channelized signals 171 generated by the filterbank module 170 in FIG. 4). The pre-processed channelized signals 1071 are provided to the F0 extractor 1082, the channel energy detector 1086, and the strategy determination module 1088. Although FIG. 10 is described with reference to the use of processed channelized signals 1071, it also to be appreciated that the operations may be performed, at least in part, using other versions of the received sound signals (e.g., the electrical input signals received from the sound input, the pre-filtered output signals, etc.).

The peak detector 1099 is configured to use the lowest frequency channel, or a broad band region, to determine pulse timing 1002 (e.g. pulse energy determined by energy either in same region as peak picker or by other region). A limit to the update rate may be determined by a fundamental frequency limit (F0_limit). The fundamental frequency limit may be 500 Hz, although this value is merely illustrative. The pulse timing 1002 is provided to the F0 imparting module 1090

As noted, the pre-processed channelized signals 1071 are provided to the channel energy detector 1086. The channel energy detector 1086 is configured to determine the energy in the lowest frequency channel, independent of whether the signal is harmonic/regular or not, and output a pitch channel energy (PCE) 1089 In one example, the channel energy detector 1086 extracts the energy in a broad band signal (e.g., the electrical input signals 167 received from the sound input devices 133 or the pre-filtered output signal 169) to drive the apical stimulation channel(s) (i.e., the F0 imparting electrode(s)). In another example, the channel energy detector 1086 extracts energy in only a low frequency range/band (e.g., below 800 Hz). The low frequency range could be adjusted on a per-recipient basis to match tonotopy e.g., by measuring acoustic pitch in contralateral car related to apical electrode and next electrode and determine appropriate frequency break point.

The F0 imparting module 1090 receives the pulse timing 1002 and the pitch channel energy 1089. The F0 imparting module 1090 is configured to analyze these values and determine pulse control parameters 1091 (e.g., pulse amplitude and timing) to encode the fundamental frequency.

The strategy determination module 1088 is configured to generate pulse control parameters 1093 (e.g., pulse amplitude and amplitude timing) in accordance with an initial pre-determined/pre-set strategy. That is, the strategy determination module 1088 uses an initial strategy to determine pulse control parameters 1093 that are configured to impart the sound information included in the received sound signals, other than the fundamental frequency information. The initial strategy used to generate the pulse control parameters 1093 may be, for example, a CIS strategy, an ACE strategy, a 500 pps strategy, etc. In certain examples, it may be possible use a lower update rate strategy which will complement the F0 imparting electrode.

The pulse control parameters 1091 to encode the fundamental frequency are provided to an arbitrator 1092. In addition to these pulse control parameters 1091, the arbitrator 1092 also receives the pulse control parameters 1093 from the strategy determination module 1088. The arbitrator 1092 analyzes the pulse control parameters 1091 and the pulse control parameters 1093 to generate stimulation control signals 1037 that represent electrical stimulation for delivery to the recipient via an apical electrode assembly and/or a basilar electrode assembly of the cochlear implant. In certain examples, the F0 imparting electrode(s) (e.g., one or more of the apical electrodes implanted in the apical region) will have high weighting in the arbitration decisions. In certain embodiments, a lower amplitude of pulse energy on the lowest frequency (pitch) channel could reduce the arbitration weight, but in general it would excerpt more control relative to other channels. That is, the arbitration decisions may be configured to be faithful to the low frequency timing, thus the arbitration process favors the low frequency channels when it determines the stimulation control signals 1037.

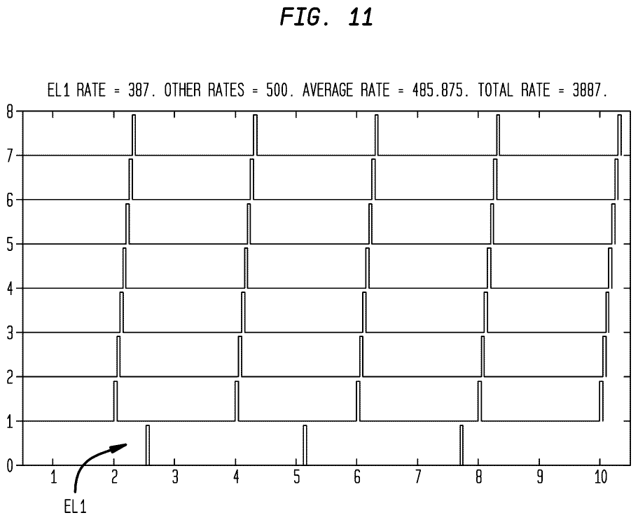

In the examples of FIGS. 8 and 10, with an F0 imparting process involving rate pitch via fundamental frequency extraction (FIG. 8) or directly via peak picking (FIG. 10), the rate on the lowest frequency channel (one or more apical electrodes of the apical electrode assembly) would follow the associated fundamental frequency. Such an example pulse train is shown in FIG. 11 where the pitch channel (EL1) represents 387 Hz with has a rate of 387 pps rate (in black at bottom). Other channels are presented at a rate of 500 pps. The average rate per channel is 486 pps.

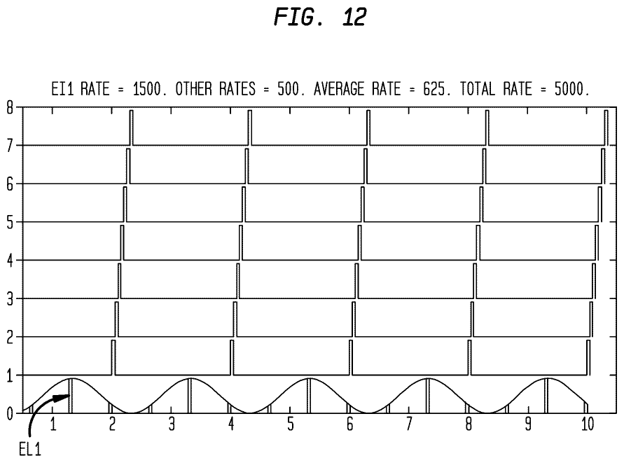

In the example of FIG. 9, with an FO imparting process involving modulation, the lowest frequency channel would impart the pitch cue. Such an example pulse train is shown in FIG. 12 where the sinusoidal amplitude modulation occurs at a high rate (500 Hz) and is reasonably represented by the 1500 pps carrier presented to the apical channel, which would not be on the other, lower rate channels. In this example, the average rate per channel is 625 pps.

In one example of the embodiment of FIG. 9, the result may be CIS-like stimulation on the lowest frequency channel with a carrier at 1500 pps, and another 8 maxima on electrodes operating at 500 pps ACE. In alternative, the result may be ACE-like stimulation on the lowest frequency channel, but could be stimulated more often.

Table 1, below, illustrates potential example rates in accordance with certain embodiments presented herein. For an eight (8) maxima presentation set, the average rate is kept lower even with one higher rate on one channel. Notice that the lowest frequency channel (EL1) at 1500 pps mixed with 500 pps is still low rate (equivalent to 8 maxima at 625 pps per channel).

TABLE 1

| | | | | Total |
|---|---|---|---|---|
| Description | El 1 Rate | Other El Rate | Avg. Rate | Rate |
| ACE 500 pps | 500 | 500 | 500 | 4000 |
| FIG. 8/FIG. 10: El1 @ 387 | 387 | 500 | 486 | 3887 |
| FIG. 9: El1 @ 1500 | 1500 | 500 | 625 | 5000 |
| ACE 900 pps | 900 | 900 | 900 | 7200 |
| ACE 1200 pps | 1200 | 1200 | 1200 | 9600 |

In summary, FIGS. 7-12 generally illustrate embodiments of a full-spectrum coordinated stimulation strategy in which the fundamental frequency (F0) of the received sound signals can be encoded into the stimulation signals delivered via one or more of the of direct low frequency channels (i.e., one or more apical electrodes of an apical electrode assembly). It is to be appreciated that encoding of the fundamental frequency is one example and that other embodiments of a full-spectrum coordinated stimulation strategy may encode other or additional frequencies, such as the second harmonic, third harmonic, etc. into the stimulation signals delivered via one or more of the of direct low frequency channels. The fundamental frequency may also be provided to more than a single electrode.

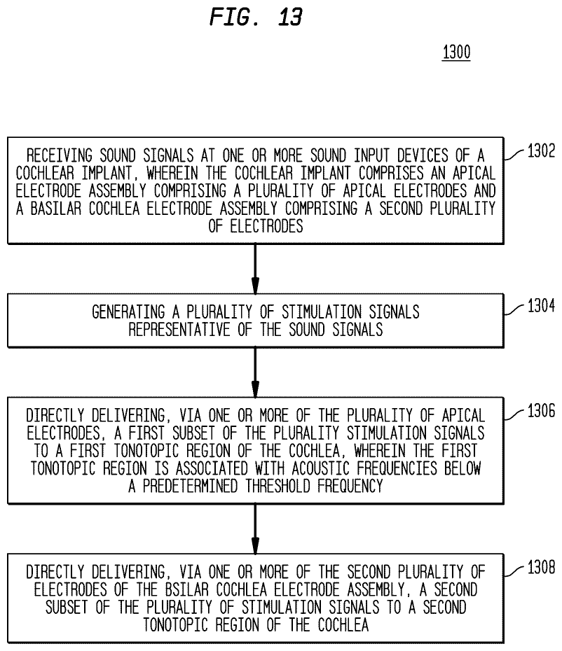

FIG. 13 is a high-level flowchart of a method 1300 in accordance with certain embodiments presented herein. Method 1300 begins at 1302 where one or more sound input devices of a cochlear implant receive sound signals. The cochlear implant comprises an apical electrode assembly comprising a plurality of apical electrodes and a basilar electrode assembly comprising a second plurality of electrodes.

At 1304, the cochlear implant generates a plurality of stimulation signals representative of the sound signals. At 1306, the cochlear implant directly delivers, via one or more of the plurality of apical electrodes, a first subset of the plurality stimulation signals to a first tonotopic region of the cochlea. The first tonotopic region is associated with acoustic frequencies below a predetermined threshold frequency. At 1308, the cochlear implant directly delivers, via one or more of the second plurality of electrodes of the basilar electrode assembly, a second subset of the plurality of stimulation signals to a second tonotopic region of the cochlea.

Individuals suffer from different types of hearing loss (e.g., conductive and/or sensorineural) and/or different degrees/severity of hearing loss. However, it is now common for many cochlear recipients to retain some residual natural hearing ability (residual hearing) after receiving a standard cochlear implant with only a basilar electrode assembly. For example, progressive improvements in the design of basilar stimulating assemblies, surgical implantation techniques, tooling, etc. have enabled atraumatic surgeries which preserve at least some of the recipient's fine inner ear structures (e.g., cochlea hair cells) and the natural cochlea function, particularly in the lower frequency regions of the cochlea.

Due, at least in part, to the ability to preserve residual hearing, not all recipients may initially be candidates for an apical cochlear implant with insertion of the apical electrode assembly because recipients with residual hearing typically benefit from having the acoustic stimulation in addition to the electrical stimulation. In these recipients, acoustic stimulation adds a more “natural” sound to their hearing perception over the electrical stimulation signals only.

However, over time, these recipients who initially retain some residual hearing may partially or completely lose this low frequency residual hearing. Therefore, in accordance with certain embodiments presented herein, an apical cochlear implant may be a type of upgrade for recipients why previously had, but have lost, acoustic hearing in the low frequency region of the cochlea. For example, when that low frequency hearing is lost, the apical electrode assembly may then be inserted and used as described elsewhere herein. In certain such examples, the apical electrode assembly may be added to a previously implanted cochlear implant (e.g., via some form of connector mechanism). In other examples, the apical electrode assembly may be placed in the recipient at the same time as the standard basilar electrode assembly (or a short basilar electrode assembly), but done in such a way that the apical electrode assembly is inactive and located outside of the cochlea (e.g., positioned along the skull bone). Other implementations are possible. In any case, once it is determined that the recipient's low frequency hearing has been lost or fallen below an acceptable threshold level, the apical electrode assembly may be inserted into the apical region of the cochlea.

The apical cochlear implant in accordance with certain embodiments presented herein may also be an upgrade for recipients of only a standard basilar electrode assembly (or short electrode assembly) who experience non-optimal device outcomes. For example, if a recipient does not experience acceptable hearing performance with their basilar electrode assembly, the apical electrode assembly may be subsequently implanted to provide access for to the lower frequencies, remove frequency shifts, etc.

It is to be appreciated that the above described embodiments are not mutually exclusive and that the various embodiments can be combined in various manners and arrangements.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1.-20. (canceled)

21. A method, comprising:
delivering, via at least one electrode of an apical electrode assembly of an implantable medical device, stimulation signals to an apical region of a cochlea of a recipient of the implantable medical device, wherein the apical electrode assembly is configured to be positioned at an apex of the cochlea; and
sinking at least a first portion of the stimulation signals via a basilar electrode assembly, wherein the basilar electrode assembly comprises a plurality of electrodes and is configured to be implanted into the cochlea of a recipient via a basal region of the cochlea.

22. The method of claim 21, wherein the apical electrode assembly comprises a plurality of electrodes.

23. The method of claim 22, wherein the method comprises:
delivering the stimulation signals via at least one electrode of the plurality of electrodes of the apical electrode assembly; and
sinking at least a second portion of the stimulation signals via one or more electrodes of the plurality of electrodes of the apical electrode assembly,
wherein the one or more electrodes are different from the at least one electrode.

24. The method of claim 23, further comprising:
delivering the stimulation signals via the at least one electrode using a focused electrode configuration where two or more of the plurality of electrodes of the apical electrode assembly sink the at least the second portion of the stimulation signals.

25. The method of claim 21, further comprising:
sinking the first portion of the stimulation signals via two or more of the plurality of electrodes of the basilar electrode assembly.

26. The method of claim 21, further comprising:
sinking at least a second portion of the stimulation signals additional an extra-cochlear electrode implanted in the recipient outside of the recipient's cochlea.

27. The method of claim 21, wherein the apical electrode assembly is physically separated and disconnected from the basilar electrode assembly.

28. The method of claim 21, further comprising:
receiving sound signals at one or more sound input devices of the implantable medical device; and
generating the stimulation signals based on the sound signals.

29. The method of claim 28, further comprising:
generating additional stimulation signals based on the sound signals;
delivering the stimulation signals to evoke perception of a first frequency range of the sound signals; and
delivering the additional stimulation signals to the cochlea via at least one of the plurality of electrodes of the basilar electrode assembly to evoke perception of a second frequency range of the sound signals.

30. The method of claim 29, further comprising:
sinking at least a portion of the additional stimulation signals via the apical electrode assembly.

31. The method of claim 29, further comprising:
band-pass filtering, via band-pass filters, the sound signals to generate a set of bandwidth limited channels each including a spectral component of the sound signals, wherein the band-pass filters have a non-uniform spectral width.

32. The method of claim 28, wherein generating the stimulation signals based on the sound signals comprises:
processing the sound signals so as to encode a fundamental frequency of the sound signals into the stimulation signals delivered via the at least one electrode of the apical electrode assembly.

33. The method of claim 32, wherein processing the sound signals so as to encode a fundamental frequency of the sound signals into the stimulation signals comprises:
setting a pulse rate of the stimulation signals delivered via the at least one electrode of the apical electrode assembly to encode the fundamental frequency of the sound signals.

34. The method of claim 32, wherein processing the sound signals so as to encode a fundamental frequency of the sound signals into the stimulation signals comprises:
setting an envelope modulation of the stimulation signals delivered via the at least one electrode of the apical electrode assembly to encode the fundamental frequency of the sound signals.

35. The method of claim 32, wherein processing the received sound signals so as to encode a fundamental frequency of the sound signals into the stimulation signals comprises:
executing a peak selection process so that the stimulation signals delivered via the at least one electrode of the apical electrode assembly encodes the fundamental frequency of the sound signals.

36. The method of claim 21, wherein delivering the stimulation signals to the apical region of the cochlea comprises:
delivering the stimulation signals to a tonotopic region of the cochlea that is associated with acoustic frequencies below approximately 2 kilohertz.

37. The method of claim 21, wherein delivering the stimulation signals to the apical region of the cochlea comprises:
delivering the stimulation signals to a tonotopic region of the cochlea that is associated with acoustic frequencies below approximately 1 kilohertz.

38. The method of claim 21, further comprising:
forming a cochleostomy in an inner ear of the recipient proximate to the apical region of the cochlea;
inserting the apical electrode assembly through the cochleostomy in the inner ear; and
inserting the basilar electrode assembly into the cochlea through an opening in the inner ear, wherein the opening in the inner ear is different from the cochleostomy.

39. A method, comprising:
sourcing a first current via an apical electrode of a plurality of apical electrodes of an apical electrode assembly, wherein the apical electrode assembly comprises a first carrier member in which the plurality of apical electrodes is disposed; and
sourcing a second current via a basilar electrode of a plurality of basilar electrodes of a basal electrode assembly, wherein the basal electrode assembly comprises a second carrier member, physically separated and disconnected from the first carrier member, in which the plurality of basilar electrodes is disposed.

40. The method of claim 39, comprising sinking the second current via at least one additional apical electrode of the plurality of apical electrodes of the apical electrode assembly.
